(19) United States
(12) Patent Application Publication
(10) Pub. No.: US 2022/0354629 A1
(43) Pub. Date: Nov. 10, 2022

(54) METHOD AND DEVICE FOR THE ENHANCEMENT OF TOPICAL TREATMENTS FOR ORAL MUCOSITIS AND OTHER ORAL CONDITIONS

(71) Applicant: E2BIO LIFE SCIENCES, LLC, Skokie, IL (US)

(72) Inventor: Eli D. Ehrenpreis, Skokie, IL (US)

(73) Assignee: E2BIO LIFE SCIENCES, LLC, Skokie, IL (US)

(21) Appl. No.: 17/868,833

(22) Filed: Jul. 20, 2022

Related U.S. Application Data
(63) Continuation of application No. 17/498,792, filed on Oct. 12, 2021.
Application No. 17/498,792 is a continuation-in-part of application No. 16/242,141, filed on Jan. 8, 2019, now abandoned.
Application No. 17/498,792 is a continuation-in-part of application No. 17/082,064, filed on Oct. 28, 2020.
Application No. 17/082,064 is a continuation-in-part of application No. 16/776,984, filed on Jan. 30, 2020.
(60) Provisional application No. 62/615,107, filed on Jan. 9, 2018.
(60) Provisional application No. 62/800,151, filed on Feb. 1, 2019.
(60) Provisional application No. 63/226,280, filed on Jul. 28, 2021.

(51) Int. Cl. A61C 19/06; A61M 31/00

(52) U.S. Cl. CPC A61C 19/063; A61M 31/002; A61M 2210/0643

(57) ABSTRACT

A method of enhancing absorption of a therapeutic agent sublingually in a person configured to treat chronic graft-versus-host disease (cGVHD) or aphthous stomatitis. The method includes administering a therapeutically effective amount of the therapeutic agent sublingually in a person and then inserting for a predetermined treatment period a device in an oral cavity of the person, wherein the device comprising an oral retention portion that is configured to be retained in the oral cavity from the predetermined treatment period and further configured to enhance absorption of the therapeutic agent sublingually.

### RELATED APPLICATION

This application is a Continuation Application of U.S. application Ser. No. 17/498,792 filed Oct. 12, 2021, which is a Continuation in Part of U.S. application Ser. No. 16/242,131 filed Jan. 8, 2019, now U.S. Pat. No. 11,229,578 issued on Jan. 25, 2022. U.S. Pat. No. 11,229,578 claims priority to U.S. provisional application Ser. No. 62/615,107, filed Jan. 9, 2018. U.S. application Ser. No. 17/498,792 is also a Continuation in Part of U.S. application Ser. No. 17/082,064 filed Oct. 28, 2020, which is a Continuation in Part of U.S. application Ser. No. 16/776,984 filed Jan. 30, 2020, which claims priority to US Provisional Application 62/800,151 filed Feb. 1, 2018. This application further claims priority to US Provisional Application 63/226,280 filed Jul. 28, 2021. All of the cited references are incorporated herein by reference.

### BACKGROUND OF THE INVENTION

Oral mucositis is an inflammation of the mucosa of the mouth that ranges from redness to severe ulceration. Also called stomatitis, mucositis is a common, debilitating complication that may be induced by, for example, chemotherapy, radiotherapy and bone marrow transplantation. Oral mucositis is found in almost 100% of patients receiving radiotherapy for head and neck tumors, in about 95% of patients undergoing bone marrow transplantation and in about 40% of patients receiving chemotherapy as it results from the effect of radiation on the oral mucosa and from the systemic effects of cytotoxic chemotherapy agents. In addition, about 90% of children with leukemia have this condition.

Patients with oral mucositis develop erythema (redness) and ulcerations of the lining of the mouth (mucosa) that result in a variety of symptoms. Other associated symptoms include diminished taste sensation (hypogeusia), noxious taste alterations (dysgeusia), complete loss of taste sensation (ageusia) and dry mouth (xerostomia).

Oral mucositis decreases quality of life, compromises nutritional status, causes weight loss and micronutrient deficiencies, and increases the risk of infections and other complications of treatment. More problematic, perhaps, oral mucositis may reduce a patient's ability to tolerate or maintain their chemotherapeutic or radiation therapy regimens. When oral mucositis develops from treatment with a specific chemotherapeutic agent, it may prevent further use of this medication, even if the treatment has the potential to save the patient's life. After completion of therapy, some patients have ongoing symptoms of oral mucositis, especially after receiving radiation therapy for cancers of the head and neck. In these patients, long-term nutritional compromise and diminished quality of life further aggravate the effects of having cancer.

The present state of treating or preventing mucositis generally involves the application of substances directly to the affected areas, as well as systemic administration. There are, however, very few effective treatments for oral mucositis. Accordingly, a need remains for better and more effective treatments for mucositis and for inhibiting or delaying the onset of mucositis.

### SUMMARY OF THE INVENTION

In one aspect of the present disclosure, a device for treating an affected area of the mucosal tissue of a person comprises: a fitting portion having an arcuate shape corresponding to the dental arche of the person; at least one covering portion defined on the fitting portion, the covering portion having a respective outer surface, an upper wing portion and a lower wing portion; at least one bite flange extending from an interior surface of the fitting portion; and an upper ledge extending from the interior surface of the fitting portion and following the arc of the fitting portion, wherein an upper surface of the upper ledge and an upper surface of the at least one bite flange are continuous with one another, and wherein the at least one covering section is more flexible than the fitting portion.

A portion of the outer surface of the covering portion may comprise a micro-textured surface having a plurality of reservoirs.

A therapeutic agent may be disposed on the outer surface of the covering portion or in the reservoirs.

In some embodiments, the therapeutic agent may be an anti-mucositis agent that comprises at least one of: a pharmaceutical; a cytoprotective agent; a mucoadhesive substance; a local anesthetic agent; or an antioxidant agent.

The at least one bite flange may further comprise a lower surface, and may be configured such that, when the device is placed in the mouth of a person, the bite flange lower surface contacts the lower teeth and the bite flange upper surface contacts the upper teeth.

Each of the upper wing portion and the lower wing portion may be thinner than the body portion and the upper wing portion may be configured to extend above a gum line of the person to cover or contact a portion of the upper inner cheek, and the lower wing portion may be configured to extend below a gum line of the person to cover or contact a portion of the lower inner cheek.

The upper and lower wing portions may be configured to cover the openings for the parotid duct and at least one salivary gland.

Another aspect of the present disclosure is a method of delivering a therapeutic agent to the mucosal tissue of a person or an affected portion thereof, which comprises: administering a therapeutically effective amount of the therapeutic agent to at least a portion of the mucosal tissue; inserting, in the mouth of the person, a device comprising: an oral retention portion that is suitably shaped to be retained in the oral cavity for a predetermined treatment period and a covering portion that has at least one surface that contacts the mucosal tissue being treated with the therapeutic agent.

Administering the therapeutically effective amount of the anti-mucositis agent may be conducted prior to chemotherapy or radiation treatment received by the person wherein the therapeutically effective amount of the anti-mucositis agent inhibits or delays the onset of mucositis.

Administering the therapeutically effective amount of the agent may comprise administering a therapeutically effective amount of an anti-mucositis agent, where the amount is provided to either treat or reduce the severity of mucositis; or to inhibit or delay the onset of mucositis.

The method may comprise: applying the agent to the portion of the mucosal tissue prior to inserting the device in the mouth of the person, applying the agent to the outer surface of the device prior to inserting the device in the mouth of the person, or, prior to inserting the device in the mouth of the person: applying a first amount of the agent to the portion of the mucosal tissue; and applying a second amount of the agent to the outer surface of the device.

The method may use a dental device that comprises: a fitting portion having an arcuate shape corresponding to the dental arche of the person and at least one covering portion, the covering portion having a respective outer surface, an upper wing portion and a lower wing portion; at least one bite flange extending from an interior surface of the fitting portion; an upper ledge extending from the interior surface of the fitting portion and following the arc of the fitting portion, wherein an upper surface of the upper ledge and an upper surface of the at least one bite flange are continuous with one another, wherein the at least one covering section is more flexible than the fitting portion, and wherein a portion of the respective outer surface of the at least one covering portion is in contact with the portion of the mucosal tissue.

A kit for delivering a therapeutic agent to the mucosal tissue of a person comprises: a therapeutically effective amount of the therapeutic agent; a device, comprising: a fitting portion having an arcuate shape corresponding to the dental arche of the person; at least one covering portion defined on the fitting portion, the covering portion having a respective outer surface, an upper wing portion and a lower wing portion; at least one bite flange extending from an interior surface of the fitting portion; and an upper ledge extending from the interior surface of the fitting portion and following the arc of the fitting portion, wherein an upper surface of the upper ledge and an upper surface of the at least one bite flange are continuous with one another, and wherein the at least one covering section is more flexible than the fitting portion; and printed instructions on how to use the device to administer the therapeutic agent to the mucosal tissue of the person.

### BRIEF DESCRIPTION OF THE FIGURES

Various aspects of the disclosure are discussed herein with reference to the accompanying Figures. It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. For purposes of clarity, however, not every component may be labeled in every drawing. The Figures are provided for the purposes of illustration and explanation and are not intended as a definition of the limits of the disclosure. In the Figures:

FIG. 1 is a front perspective view of a mucositis treatment device, in accordance with an aspect of the present disclosure;

FIG. 2 is a rear perspective view of the device of FIG. 1;

FIG. 3 is a top view of the device of FIG. 1;

FIG. 4 is a rear perspective view of the device of FIG. 1;

FIG. 5 is a magnified rear perspective view of a portion of the device of FIG. 1;

FIG. 6 is a front view of the device of FIG. 1;

FIG. 7 is a front perspective view of a mucositis treatment device, in accordance with another aspect of the present disclosure;

FIG. 8 is a rear perspective view of the device of FIG. 7;

FIG. 9 is a magnified rear perspective view of a portion of the device of FIG. 7;

FIG. 10 is an inside view of the right portion of the device of FIG. 10 which includes a covering portion for the front and lateral portions of the tongue;

FIG. 11 is an inside view of the right portion of the device of FIG. 10 which also shows the portion that covers the area beneath the tongue and floor of the mouth inside of the teeth;

FIG. 12 is a posterior superior view of the device of FIG. 11;

FIG. 13 is a posterior superior view of the device of FIG. 10;

FIG. 14 is a posterior view of the device of FIG. 10;

FIG. 15 is a posterior view of the device of FIG. 11;

FIG. 16A is a graph that shows the results of an oral retention test in accordance with aspects of the present disclosure;

FIG. 16B is a graph that shows the results of an oral retention test, with respect to a subject's perception of numbness, in accordance with aspects of the present disclosure;

FIGS. 17A and 17B demonstrate the results of experiments showing the effect of the oral treatment device 200 device on sublingually administered substances;

FIGS. 18A and 18B show the results of a study measuring ETOH levels in the breath after a mixture of CBD oil and alcohol is placed under the tongue using a medicine dropper;

FIG. 19 illustrates a partial mouth guard showing a phalanx in accordance with one embodiment of the invention;

FIG. 20 illustrates a mouth guard with a dental arch in accordance with one embodiment of the invention;

FIG. 21 illustrates a mouth guard with a front wing portion in accordance with one embodiment of the invention;

FIG. 22 illustrates a mouth guard with upward and downward extending exterior surfaces in accordance with one embodiment of the invention;

FIG. 23 illustrates a device having inner covering portions fitted between the inside teeth and roof and floor of the mouth in accordance with one embodiment of the invention; and

FIG. 24 illustrates a device having wings covering the ostia of the parotid glands and portions of the sublingual glands in accordance with one embodiment of the invention.

### DETAILED DESCRIPTION

In the following detailed description, details are set forth in order to provide a thorough understanding of the aspects of the disclosure. It will be understood by those of ordinary skill in the art that these may be practiced without some of these specific details. In other instances, well-known methods, procedures, components and structures may not have been described in detail so as not to obscure the aspects of the disclosure.

It is to be understood that the present disclosure is not limited in its application to the details of construction and the arrangement of the components or steps set forth in the following description or illustrated in the drawings as it is capable of implementations or of being practiced or carried out in various ways. Also, it is to be understood that the phraseology and terminology employed herein are for description only and should not be regarded as limiting.

Certain features are, for clarity, described in the context of separate implementations and may also be provided in combination in a single implementation. Conversely, various features, that are, for brevity, described in the context of a single implementation, may also be provided separately or in any suitable sub-combination.

A major disadvantage of existing mucositis therapies is that they remain at the site of application for a very short period of time, typically about 10 minutes or less. Treatments applied to the oral mucosa in the forms of gels, mouthwashes, ointments, etc., are quickly diluted by oral saliva and are subsequently swallowed by the patient, resulting in very short contact time with the areas affected by oral mucositis. Thus, treatments do not remain concentrated at the areas that are affected by oral mucositis and are systemically absorbed, reducing their efficacy for treating the condition and increasing their potential for systemic toxicity.

Broadly, as described herein, aspects of the present invention provide a device and method of treating diseases or disorders that affect the oral cavity and which are amenable to treatment with locally applied therapeutic agents (also referred to herein as active agents, drugs or medicaments). In some embodiments, the device and method are useful for treating oral mucositis that may entail delaying, inhibiting the onset of, or reducing the severity or duration of mucositis. A combination of the active (e.g., anti-mucositis) agent (also referred to herein as medication or treatment) and the device increases the retention time of the agent on the affected mucosal tissue. The increased retention time may enhance the efficacy of the agent. In addition, by covering and partially blocking the ostia of the parotid glands and some portions of the sublingual glands, salivary dilution of topical agents, particularly the portion of the medication that is adherent to the tongue is decreased.

In some embodiments the agent may be administered topically in the mouth, followed by placement of the device that is brought into direct contact with and covers the treated mucosal tissue. In other embodiments, the medication may be applied to, or coated onto, the device beforehand. This approach may also provide a more targeted delivery of the agent to the affected mucosal tissue, as well as an increase in retention time. In yet other embodiments, the medication may be applied to the affected tissue, followed by insertion of the device having additional medication dispersed therein.

In addition to oral mucositis, aspects of the present invention are directed toward treatments of other oral diseases or disorders such as leukoplakia, i.e., precancerous lesions in the mouth and primary xerostomia.

The device, in accordance with aspects of the present disclosure set forth herein, can be comfortably held in the mouth for prolonged periods of time and therefore extend the contact time of an active (e.g., anti-mucositis) agent on the oral mucosa. Thus, aspects of the present disclosure may provide one or more advantages such as an increase in the concentration and duration of contact of the active (e.g., anti-mucositis) agent with the area(s) of mucosa that is/are, or could be, affected with the oral disease or disorder such as oral mucositis; a decrease in systemic toxicity of the active agents as the agents may be administered in lower doses that will remain within the oral cavity longer and will, therefore, have lower systemic absorption from being swallowed; and further an increase in topical pharmacologic activity at the site of release, i.e., the oral mucosa, compared to conventional methods of direct delivery of substances onto the oral mucosa such as spray and gel forms of these medications. Still further, aspects of the present disclosure may diminish salivary dilution of topical treatments by covering and partially blocking the ostia of the parotid glands and some of the salivary gland ostia.

Broadly, the present inventive methods utilize a combination of an active agent and a device that increases the retention time of the agent on the affected mucosal tissue. The increased retention time may enhance the efficacy of the agent. In some embodiments the agent may be administered topically in the mouth, followed by placement of the device that is brought into direct contact with and covers the treated mucosal tissue. In other embodiments, the medication may be applied to or coated onto the device beforehand. This approach may also provide a more targeted delivery of the agent to the affected mucosal tissue, as well as increase retention time. In yet other embodiments, the medication may be applied to the affected tissue, followed by insertion of the device having additional medication dispersed therein.

Devices suitable for use in the present methods may have several individual components. The first component of the device is an oral retention or fitting portion that functions to comfortably hold the device in the mouth by fitting over the teeth where it can be held for a predetermined period of time or treatment. The fitting portion of the device may fit over the upper teeth, the lower teeth, both the upper and lower teeth or a portion of either or both the upper and lower teeth. This portion of the device may be preconstructed to fit any size mouth. Alternatively, it may be made of a pliable material that can be fitted to the specific patient, either by the patient or by a medical professional such as a dentist. This portion of the device may be constructed of materials that will fit the needs of the device in terms of biocompatibility, ease of device cleaning, and the stability of the material within the mouth. The fitting portion may be made using materials, e.g., poly (methyl methacrylate), polyurethane, and co-polymers of vinyl acetate or ethylene. Other materials include polyvinyl acetate-polyethylene or ethylene vinyl acetate (EVA) copolymer, polyvinylchloride, latex rubber, acrylic resin and other laminated or non-laminated thermoplastics. In some embodiments, the materials for construction may also include a dual layering of a soft material on the inside portion and a hard acrylic outside portion that holds the device firmly to the teeth and forms a protective shell for the device.

One or more other covering portions of the device are designed to fit or cover at least the major parts of the mouth that are affected by the oral disease or disorder. These covering portions of the device may be constructed of soft materials that rest gently along the mucosa (lining) of the cheeks (called the buccal mucosa), the inner portions of the lips, the palate and the mucosa underneath the tongue. These portions of the device may be constructed of materials such as latex, synthetic polyisoprene, nitrile, polyurethane resin, plastic, polyester, acrylic resin pads, silicone, polypropylene, low density polyethylene, and various laminates or layered soft/soft laminates. In some embodiments, laminates may feature a top layer of denser vinyl for memory and abrasion resistance and a soft pliable bottom layer for patient comfort.

The covering portion of the device may include an additional layer of semi-porous material that will be in contact with the oral mucosa. This will allow for anti-mucositis agents to be placed or disposed on the device, so they may be gradually released onto the affected tissue. Representative examples of materials that may be used for this part of the device include materials used for wound dressings such as thin, semi-permeable polyurethane films coated with a layer of acrylic adhesive. Other materials, such as gels may be incorporated into the mucosal (i.e., inner) side of the covering portion of the device. The use of gels allows a soft, generally water-soluble material to adhere to this portion of the device. Active agents such as antibiotics and local pain-relieving drugs can be mixed into or incorporated into the matrix of the gels. Examples of such gels include carboxymethylcellulose-based gels and alginate-based gels. In some embodiments, the inner portion may include foam-like materials. Foam materials provide a soft interface with the affected oral mucosa. Active agents may be held within the matrix of these foam-like materials. In addition, the material used for the internal layer of the covering portion of the device that is in contact with the mucosa may include drug-eluting fibers. These may include monolithic polymer fibers and reservoir fibers such as polylactic acid (PLLA) in which the drug is dissolved or dispersed. Other materials that may be used for the internal portion of this part of the device include nano porous materials such as ceramics, composites, metals, and polymeric organic substances. Other examples of materials include nano porous oxides, including alumina, titania, silica, zirconia, polycarbonate, polyethylene terephthalate, polysulfide and polymers combined with ceramics. Nano porous materials may be produced by anodization, lithography, focused ion beam etching, ion-tracking etching, phase separation and sol-gel processes.

Thus, in some embodiments, the device may have an upper part that fits on upper teeth (e.g., upper front teeth) and has a covering portion that drapes across the palate and the inner part of the upper lip. The device may have a lower part that fits on lower teeth (i.e., lower front teeth) and has a covering portion that drapes across the sublingual mucosa and inner portions of the cheek (the buccal mucosa). Devices may also have adjustable components that allow the device to fit snugly and comfortably in the mouth of an affected patient. Thus, both the upper and lower parts of the device may contain a system that allows for adjustment of these portions of the device to fit comfortably in the mouth. Adjustments can be made to shorten or lengthen the device as well as to elevate of lower the device to assist with fitting of the apparatus for comfortable and prolonged use.

Additional modifications of the device may be made in order to optimize fitting in the mouth and for individualizing the construction of the device based on the patient's oral anatomy. Such modifications may be made with the use of imaging methods such as radiographic procedures and 3-dimensional photographic imaging. Furthermore, the use of dental-type molds may be used to optimize sizing of the device for individual patients. Yet further modifications may include addition of materials such as padding and anchoring portions of the device to add to the comfort level of its use. Other modifications may be made based on methods to size the individual portions of the device to optimize covering of the affected area of the oral mucosa. Further modifications may be performed to allow the device to be worn overnight.

The active agents may be applied to the covering portion(s) of the device that come into contact with the affected mucosal tissue. The use of semi-porous materials to line these portions of the device, imparts sustained release properties to the device which allow for gradual and prolonged exposure of the mucosa to the agents. In some embodiments, the medications may be first applied to the affected area(s), followed by placement of the device in the mouth. Using this method, the device holds the medications in place and allows their prolonged contact with the mucosa.

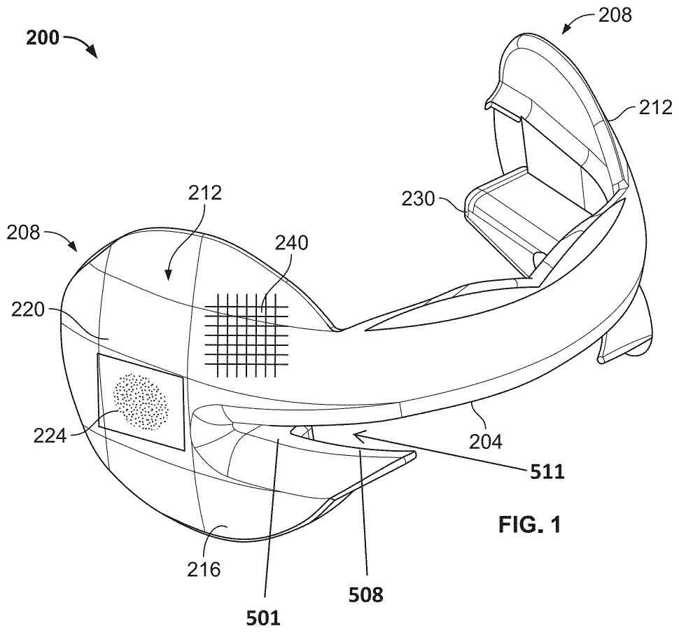

An oral treatment device 200, as shown in FIG. 1, in accordance with an aspect of the present disclosure, is suitable for use with the methods described herein. The device 200 may have several individual components or portions, however, it should be noted that “component” does not necessarily mean a separate piece or pieces unless specifically set forth as such. The device 200 includes a curved, or arcuate, oral retention or fitting portion or body 204 that can be comfortably held for a predetermined period of time or treatment. The fitting portion 204 of the device 200 has an arc that generally corresponds to the arrangement of teeth in the human mouth, i.e., the dental arche, and may fit over the upper teeth, the lower teeth, both the upper and lower teeth or a portion of either or both the upper and lower teeth.

The device 200 may be preconstructed to fit any size mouth. Alternatively, it may be made of a pliable material that can be fitted to the specific patient, either by the patient or by a medical professional such as a dentist. The device 200 may be constructed of materials that meet biocompatibility requirements for human use, ease of device cleaning and the stability of the material within the mouth. The device 200 may be made using materials including, e.g., poly (methyl methacrylate), polyurethane and co-polymers of vinyl acetate or ethylene. Other materials that could be used include polyvinyl acetate-polyethylene or ethylene vinyl acetate (EVA) copolymer, silicone rubber, polyvinylchloride, latex rubber, acrylic resin and other laminated or non-laminated thermoplastics.

The device 200 includes symmetrically provided covering portions 208, each having an upper wing portion 212 and a lower wing portion 216 configured to fit or cover parts of the mouth that are affected by mucositis. These covering portions 208 of the device 200 are generally more flexible (e.g., compliant or softer) than the body portion 204 by, for example, being thinner than the body portion 204 or being of a different material. The covering portions 208 rest along the sensitive mucosa (lining) of the cheeks (called the buccal mucosa), the inner portions of the lips, the palate and the mucosa underneath the tongue.

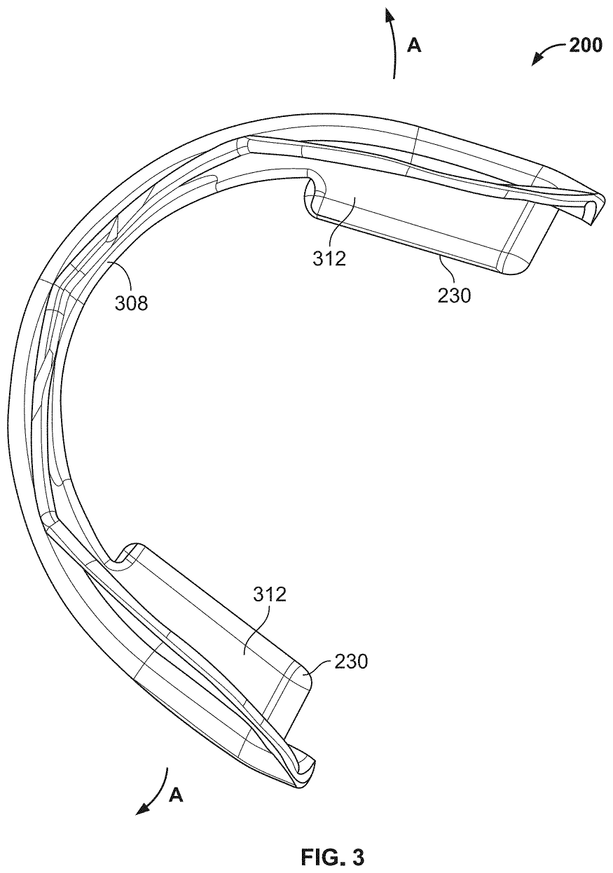

These covering portions 208 of the device 200, in one aspect of the present disclosure, may be constructed of materials, in addition to those materials listed above, such as latex, synthetic polyisoprene, nitrile, polyurethane resin, silicone rubber, plastic, polyester, acrylic resin pads, silicone, polypropylene, low density polyethylene and various laminates or layered soft/soft laminates. In some embodiments, laminates may feature a top layer of denser vinyl for memory and abrasion resistance and a soft pliable bottom layer for patient comfort. Further, the choice of materials for the device 200 provides a predetermined amount of “spring load” that urges the covering portions 208 outward as represented by the arrows A in FIG. 3.

The covering portions 208 of the device 200 have an exterior surface 220 that may include an additional layer 224 of semi-porous material that will be in contact with the oral mucosa. This semi-porous layer 224 will allow for anti-mucositis agents to be placed or disposed on the device 200, in order to be gradually released onto the affected tissue. Representative examples of materials that may be used for this part of the device include materials used for wound dressings such as thin, semi-permeable polyurethane films coated with a layer of acrylic adhesive.

Other materials, such as gels, may be incorporated onto the exterior surface 220 of the covering portion 208 of the device 200. The use of gels allows a soft, generally water-soluble material to adhere to this portion of the device 200. Medications such as antibiotics and local pain-relieving drugs can be mixed into or incorporated into the matrix of the gels. Examples of such gels include carboxymethylcellulose-based gels and alginate-based gels.

Referring again to FIG. 1, each covering portion 208 and its respective upper and lower wing portions 212, 216 are made of softer or more flexible material, as set forth above. The upper wing portions 212 extend above the upper gum line to cover or contact a portion of the upper inner cheek, and the inner upper lip, while the lower wing portions 216 extend below the lower gum line to cover or contact a portion of the lower inner cheek. In addition, the upper and lower wing portions 212, 216 cover the openings for the parotid duct and a number of salivary glands.

In one embodiment of the device 200, a portion of the external surface 220 may be treated to provide a micro-textured surface 240 that results in small reservoirs being created, as shown in FIG. 1. The micro-textured surface 240 may be provided in the device 200 by etching a pattern into the mold that is used to make the device 200. Alternatively, the external surface 220 of the device 200 may be modified by etching or sandblasting once freed from the mold. These small reservoirs serve to retain in place therapeutic materials that have been applied to the inner cheek to treat mucositis. Advantageously, the medicine is then in place longer before it is washed away by saliva. Alternatively, the therapeutic material may be “loaded” onto the micro-textured surface 220 before the device 200 is placed in the mouth which may aid in application to the inner mucosa.

In some embodiments, the covering portion 208 may include foam-like materials. Foam materials provide a soft interface with the affected oral mucosa. Medications may be held within the matrix of these foam-like materials.

In addition, the material used for the exterior surface 220 of the covering portions 208 of the device 200 that is in contact with the mucosa may include drug-eluting fibers. These fibers may include monolithic polymer fibers and reservoir fibers such as polylactic acid (PLLA) in which the drug is dissolved or dispersed.

Still further, other materials that may be used on the exterior surface 220 of the covering portion 208 include nano porous materials such as ceramics, composites, metals and polymeric organic substances. Other examples of materials include nano porous oxides, including alumina, titania, silica, zirconia, polycarbonate, polyethylene terephthalate, polysulfide and polymers combined with ceramics. Nano porous materials may be produced by anodization, lithography, focused ion beam etching, ion-tracking etching, phase separation and sol-gel processes.

The body portion 204 includes an upper portion configured to cover the mucosa between the upper teeth and the gums and the upper inner lip. Advantageously, this aids in retention of the device 200 within the mouth by holding it in place between the upper inner lips and upper teeth. This portion is constructed of thinner or more pliable material for comfort and for holding medication in place in that area of the mouth.

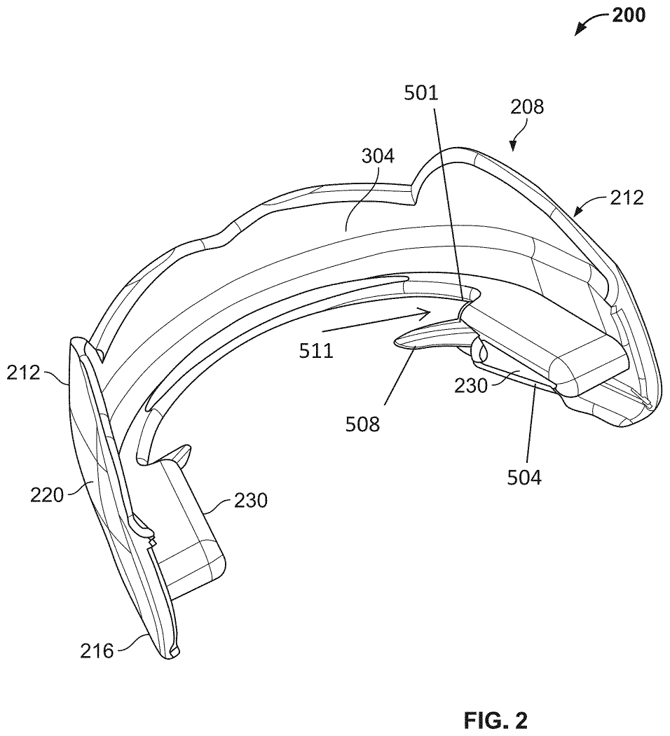

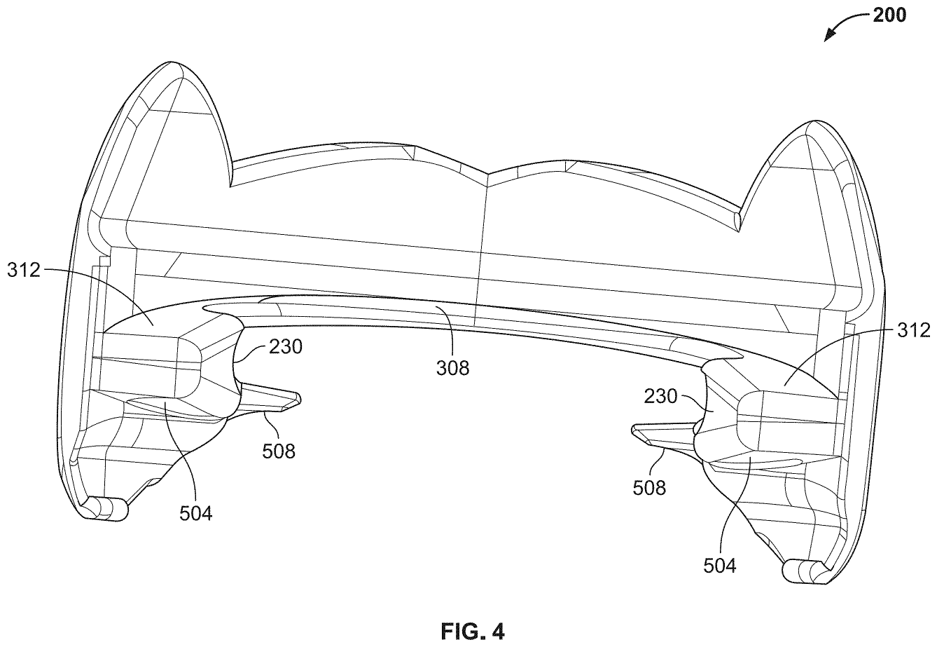

Referring to FIGS. 2 and 4, each of which is a rear perspective view, the device 200 may include symmetrically opposed bite flanges 230 that extend from an interior surface 304. In addition, an upper ledge 308 also extends from the interior surface 304and when the device 200 is placed in the mouth of a patient, the upper teeth of the patient may rest thereon.

As shown in FIG. 3, which is a top view of the device 200, the upper ledge 308 is continuous around an inner arc of the device 200 and is contiguous with an upper bite surface 312 of the bite flanges 230. Each bite flange 230 includes a lower bite surface 504 that, in conjunction with the upper bite surface 312, forms a wedge shape. When the device 200 is placed in the patient's mouth, the top teeth will rest on the upper ledge 308 and some of the upper and lower back teeth, e.g., mid-molars, can bite down on the upper and lower bite surfaces 312, 504 of the bite flange 230. In this manner, the device 200 can be held in place with minimal effort. The bite flange 230 is made of softer or spongier material that the patient can bite down on without becoming fatigued too quickly.

As shown in FIGS. 1 and 2 and in FIG. 3, which is a top view of the device 200, the upper ledge 308 is continuous around an inner arc of the device 200 and is contiguous with an upper bite surface 312 of the bite flanges 230. Each bite flange 230 includes a lower bite surface 504 that, in conjunction with the upper bite surface 312, forms a bite wedge 231. When the device 200 is placed in the patient's mouth, the top teeth will rest on the upper ledge 308 and some of the upper and lower back teeth, e.g., mid-molars, can bite down on the upper and lower bite surfaces 312, 504 of the bite flange 230. In this manner, the device 200 can be held in place with minimal effort. The bite flange 230 is made of softer or spongier material that the patient can bite down on without becoming fatigued too quickly. In addition, each lower wing portion 216 further includes a v-shaped notch 501 being positioned adjacent each bite wedge 231 towards the middle section 204. Wherein, the lower surface edge along the middle section of the fitting portion extends arcuately towards each lower wing portion the v-shaped notch 501 defines a gap 511 between the lower surface edge and the lower tab 508 extending from the lower bite flange 504.

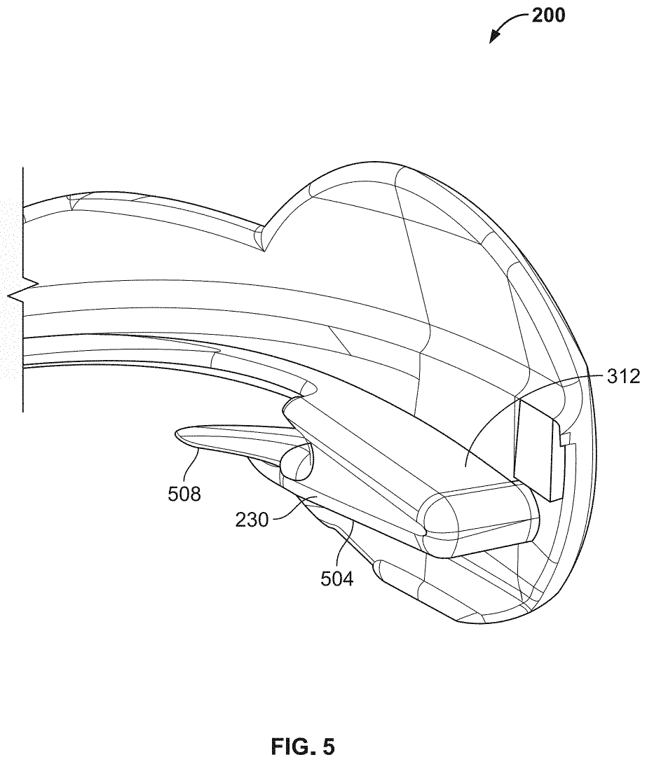

As shown in FIGS. 4 and 5, and as seen from the back of the device 200, symmetric lower tabs 508 are provided forward of each respective bite flange 230. The lower tabs 508 rest on some of the lower teeth forward of those that are biting down on the bite flanges 230. The lower tabs 508 provide additional stability and absorb some stress from the patient holding on, i.e., biting down, on the bite flanges 230.

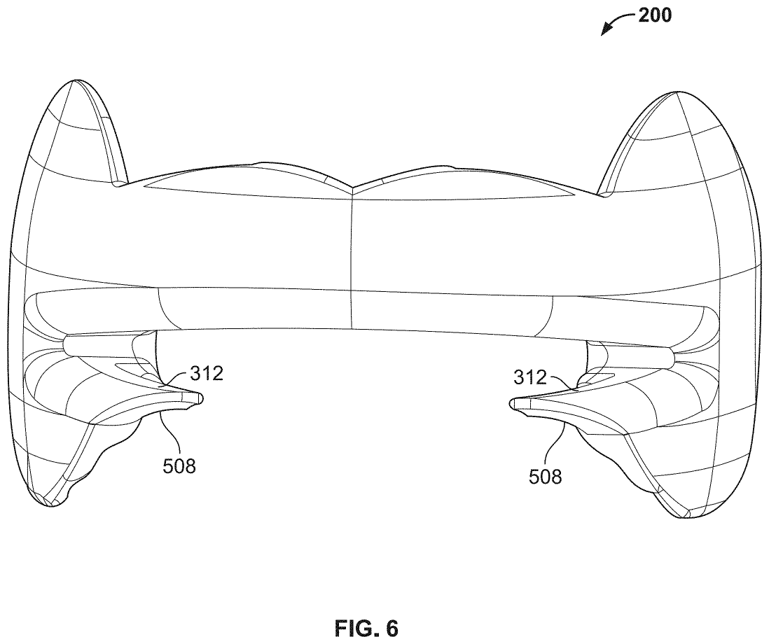

As shown in FIG. 6, which is a front view of the device 200, the lower tabs 508 may extend around a portion of the arc of the lower teeth. In another embodiment, the lower tabs 508 may extend all the way around the arc into a single lower tab, i.e., a continuous piece for all of the lower teeth to contact.

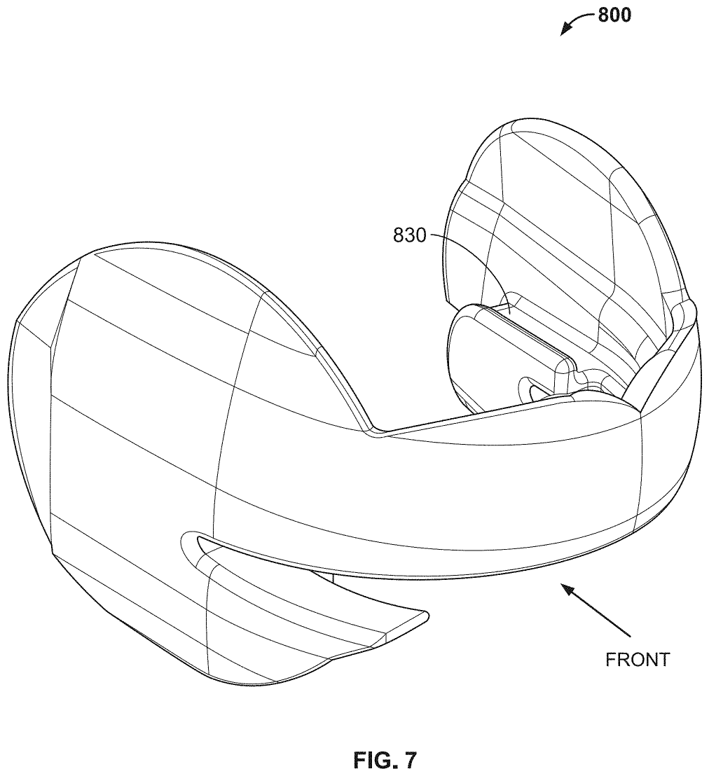

Referring now to FIG. 7, another mucositis treatment device 800, in accordance with an aspect of the present disclosure that is suitable for use with the methods described herein is presented from a front perspective view. The device 800 is similar to the device 200 as set forth above, however, the device 800 includes symmetrically opposed bite flanges 830 that differ from the bite flanges 230 described above.

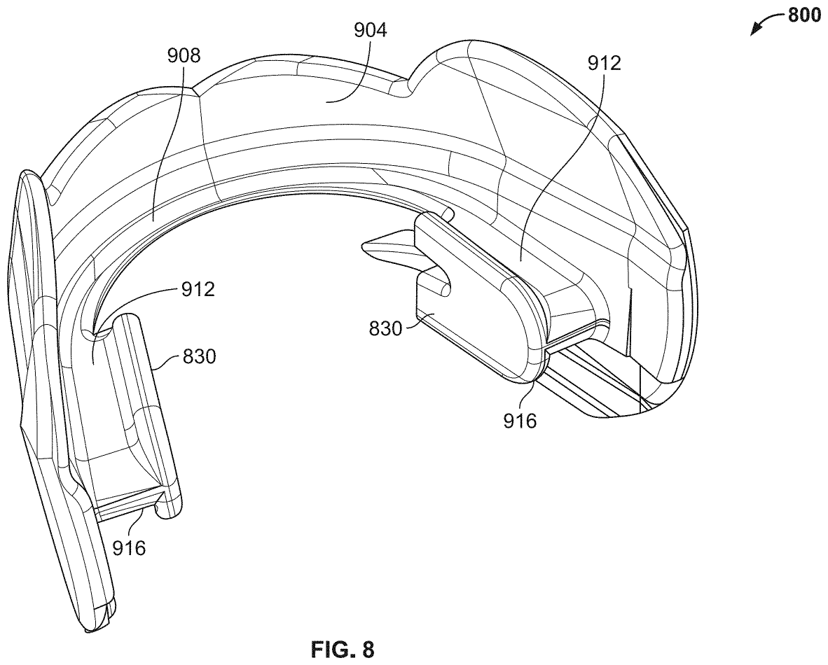

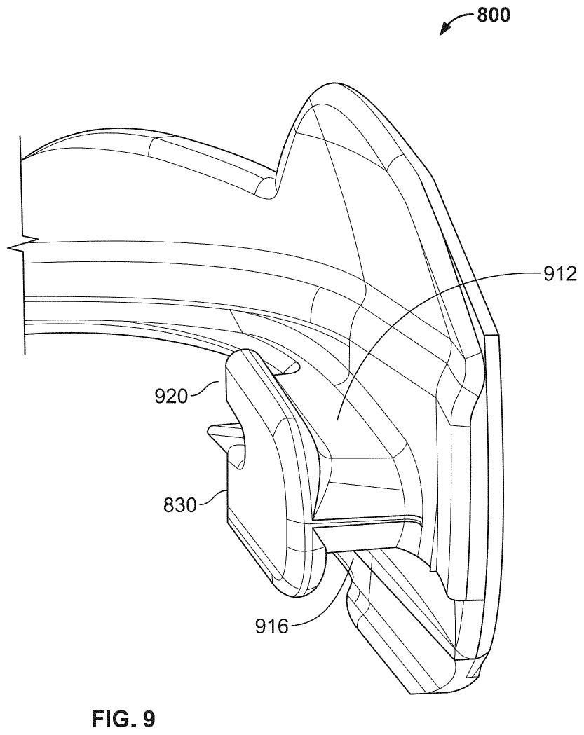

As shown in FIGS. 8 and 9, the bite flanges 830 extend from an interior surface 904. An upper ledge 908 also extends from the interior surface 904 and when the device 800 is placed in the mouth of a patient, the upper teeth of the patient may rest thereon. The upper ledge 908 is continuous around an inner arc of the device 800 and is contiguous with an upper bite surface 912 of the bite flanges 830. Each bite flange 830 includes a lower bite surface 916. When the device 800 is placed in the patient's mouth, the top teeth will rest on the upper ledge 908 and some of the upper and lower back teeth, e.g., mid-molars, can bite down on the upper and lower bite surfaces 912, 916 of the bite flange 830. In this manner, the device 800 can be held in place with minimal effort. Each bite flange 830 is made of softer or spongier material that the patient can bite down on without becoming fatigued too quickly.

In addition to the upper and lower bite surfaces 912, 916, the bite flange 830 includes a guide ridge 920 that is generally transvers to the upper and lower bite surfaces 912, 916. The guide ridge 920 provides a space to line up the upper teeth in order to maintain the device 800 in position. Advantageously, the patient need not bite down on the bite flange 830 to keep the device 800 in place in the mouth with the side portions positioned against the inner cheeks.

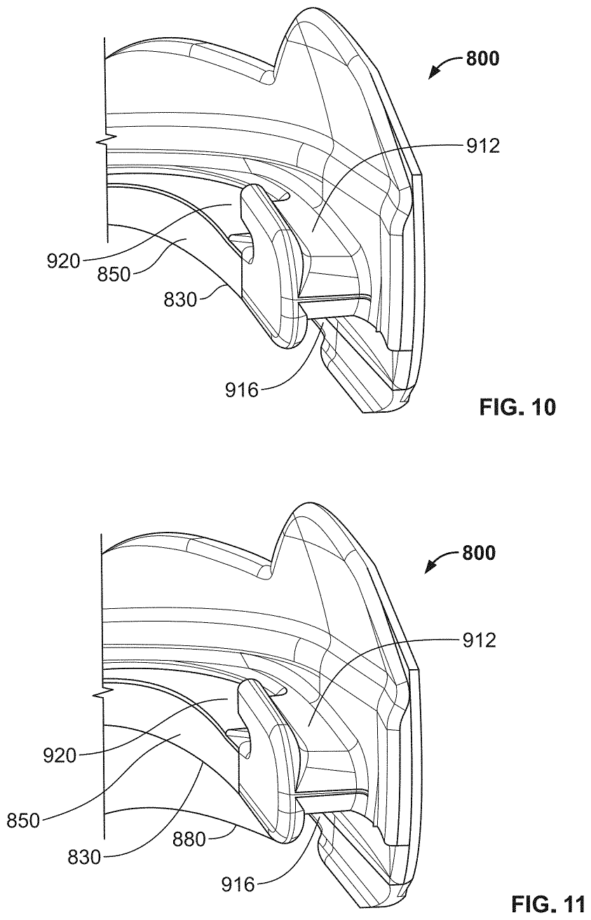

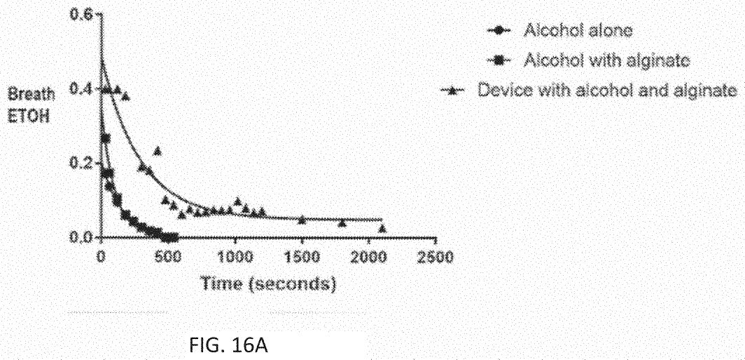

FIGS. 10-15 illustrate a device that is a modified version of FIG. 7, and which includes a covering portion for the front and lateral portions of the tongue. FIG. 10 is an inside view of the right portion of the device. This form of the device includes a soft covering portion (850) for the lateral and front of the tongue that follows the arcuate design of the device. This soft covering portion for the lateral tongue is attached to the inferior portion of the bite flange (830), is a thicker depth than the covering wings. Part 850 rests comfortably and wraps around the lateral portions of the tongue, forming a covering over the sides and front of the tongue.

FIG. 11 is an inside view of the right portion of the modified device. This form of the device includes a soft covering portion 850 for the lateral and front of the tongue that is attached to the inferior part of the bite flanges 830 and follows the arcuate design of the device. It also shows the portion 880 that covers the area beneath the tongue and floor of the mouth inside of the teeth. This is a winglike portion of the device with similar thin structure and fits under the tongue. It is attached to both the inferior portion of the bite flanges 830 and to the inferior portion of the lateral tongue covering portion 850 of the device. The tongue can be slipped into combined portions 850 and 880, thus providing coverage of the lateral tongue, the ventral tongue, and the mucosa of the floor of the mouth inside of the teeth.

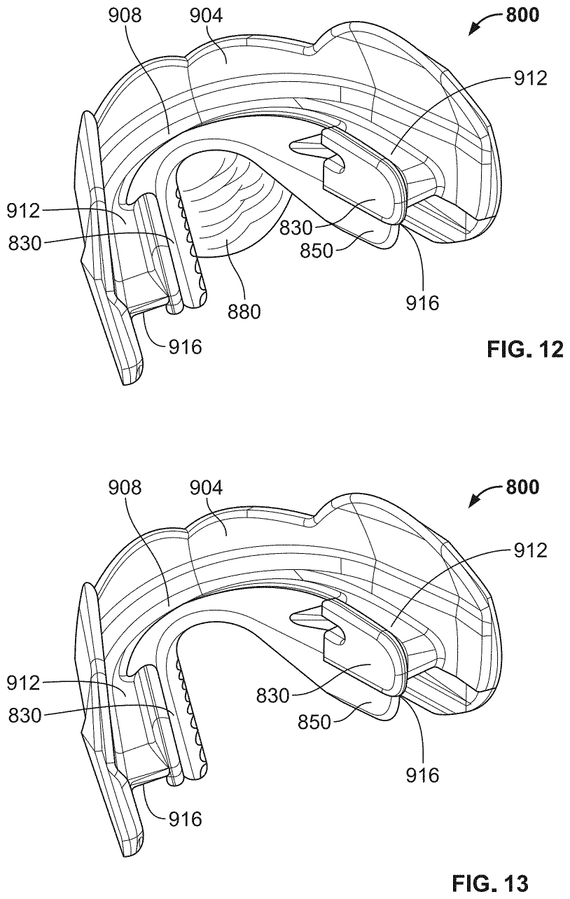

FIG. 12 is a posterior superior view of the device of FIG. 11. This view demonstrates the soft covering portion 850 for the later and front of the tongue that is attached to the inferior part of the bite flanges 830 and follows the arcuate design of the device. It also shows the portion 880 that covers the area beneath the tongue and floor of the mouth inside of the teeth. This is a wing-like portion of the device and has a similar thin structure to the cheek and inner lip covering wings and fits under the tongue. It is attached to both the inferior portion of the bite flanges 830 and to the inferior portion of the lateral tongue covering portion 850 of the device.

FIG. 13 is a posterior superior view of the device of FIG. 10. This view shows the soft covering portion 850 for the lateral and front of the tongue that is attached to the inferior part of the bite flanges 830 and follows the arcuate design of the device.

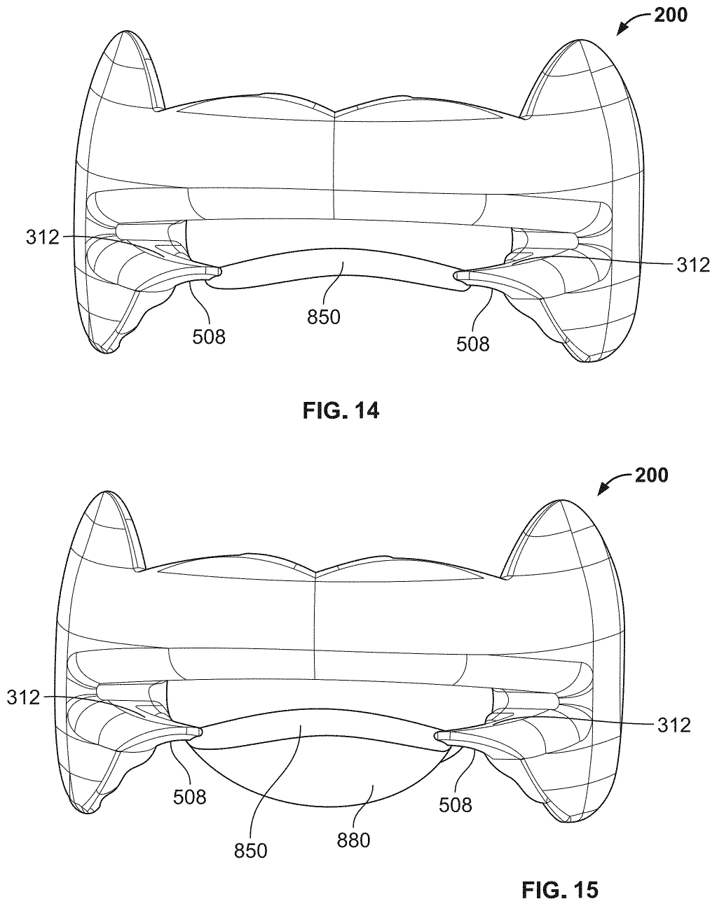

FIG. 14 is a posterior view of the device of FIG. 10. This view demonstrates the soft covering portion 850 for the lateral and front of the tongue that is attached to the inferior part of the bite flanges 830 and follows the arcuate design of the device. This design allows portion 850 to easily rest on the sides and front of the tongue.

Additional modifications of the device may be made in order to optimize fitting in the mouth and for individualizing the construction of the device based on the patient's oral anatomy. Such modifications may be made with the use of imaging methods such as radiographic procedures and 3-dimensional photographic imaging. Furthermore, the use of dental-type molds may be used to optimize sizing of the device for individual patients. Yet further modifications may include addition of materials such as padding and anchoring portions of the device to add to the comfort level of its use. Other modifications may be made based on methods to size the individual portions of the device to optimize covering of the affected area of the oral mucosa. Further modifications may be performed to allow the device to be worn overnight.

Advantageously, the designs of the devices described above follow the contour of the mouth for comfort. The bite flanges provide surfaces for both the upper and lower teeth to close on and hold the device in place. In one aspect, the device is configured to be located over only the upper teeth as this provides additional comfort compared to devices that fit over both the upper and lower teeth, in addition to making it easier for the patient to breathe while the device is in place. Still further, a device where only the upper teeth are covered allows for easy elimination of saliva without the necessity to remove the device.

The side portions between teeth, gums and inner lips provide coverage of gums, upper oral mucosa and inside of upper lip and a portion of the lower lip allowing for increased mucosal coverage and which also helps to hold the device in place.

The active (e.g., anti-mucositis) agents may be applied to the covering portion(s) of the device that come into contact with the affected mucosal tissue. The use of semi-porous materials to line these portions of the device, imparts sustained release properties to the device that allow for gradual and prolonged exposure of the mucosa to the agents. In some embodiments, the medications may be first applied to the affected area(s), followed by placement of the device in the mouth. Using this method, the device holds the medications in place and allows their prolonged contact with the mucosa.

When the device is held within the mouth, the anti-mucosal agents remain in contact with the oral mucosa that is affected by mucositis for a predetermined time or treatment period which for purposes of the present invention, may include a contact time of 10 minutes, 20 minutes, 30 minutes, 60 minutes, 90 minutes, 120 minutes, 150 minutes, 180 minutes, 210 minutes, 240 minutes or more. In some embodiments, methods entail the device being held in the mouth for about 10 minutes to up to 2 hours at a time. The method may be conducted multiple times per day, for example, but not limited to, from 1-4 times daily.

Broadly, any agent that exerts a therapeutic or prophylactic effect when topically applied to mucosal tissue may be suitable for use in the present invention, types of which include mucosal-adherent substances, mucosal protectant agents, antioxidants, antibiotics and oral analgesics. Representative examples of both classes of agents and specific agents that may be suitable for use in the present invention are listed in Table 1 and natural substances for treatment of oral mucositis and other oral conditions are listed in Table 2. Representative examples of mucoadhesive polymers that may also be used in accordance with aspects of the present disclosure, are listed in Table 3. Treatment of oral mucositis may also entail use of the compositions listed in Table 4.

TABLE 1
Pharmaceuticals

| | |
|---|---|
| 1) | Benzydamine mouthwash |
| 2) | Doxepin mouthwash |
| 3) | 0.2% morphine mouthwash |
| 4) | Palifermin, a truncated human recombinant keratinocyte |
| | growth factor (KGF) |

Antioxidant Agents

| | |
|---|---|
| 1) | Glutamine |
| 2) | Oral Zinc Supplement |
| 3) | Vitamin E |
| 4) | N-Acetyl-Cysteine (NAC) |
| 5) | Superoxide Dismutase Mimetics |

Inflammation and Cytokine Production-Inhibitors

| | |
|---|---|
| 1) | Production-Inhibitors |
| 2) | Pentoxifylline |
| 3) | Salicylates |
| 4) | Interleukin Inhibitors |

Other Biological Modifiers in Development

| | |
|---|---|
| 1) | Smad7 inhibitors |
| 2) | TGFb inhibitors |
| 3) | NF-kB inhibitors |
| 4) | Dusquetide- an innate immune inhibitor |
| 5) | Topical formulation of clonidine |
| 6) | Trefoil factor 1 released by genetically modified |
| | Lactococcus lactis bacteria |
| 7) | EC-18, primarily directed at neutropenia |

Cytoprotective Agents

| | |
|---|---|
| 1) | Prostaglandin analogs |
| 2) | Sucralfate in various formulations |

Growth Factors

| | |
|---|---|
| 1) | Palifermin |
| 2) | Granulocyte-macrophage colony-stimulating factor(GM-CSF), |
| 3) | Granulocyte colony-stimulating factors (G-CSF, e.g., filgrastim) |
| 4) | FGF |

Antiapoptotic Agents

| | |
|---|---|
| 1) | Anti-chemokine ligand 9 (CXCL9) |
| 2) | Specific caspase-3 inhibitors |

Probiotics including Lactobacillus species
Antibiotics

| | |
|---|---|
| 1) | Chlorhexidine |
| 2) | Other topical antibiotics |

Other Agents

| | |
|---|---|
| 1) | Glucagon-Like Peptide-2 (GLP-2) analogs |
| 2) | Vitamin A |
| 3) | Vitamin E |
| 4) | Ascorbic acid |
| 5) | Chamomile |
| 6) | Curcumin |
| 7) | Other plant extracts |

Topical Mucosal Protectant Agents

| | |
|---|---|
| 1) | Sucralfate |
| 2) | Polymerized sucralfate in pastes and other forms |
| | including ProThelial ™ |
| 3) | Oral mucoadhesive hydrogels including MuGard ™ |
| 4) | Polyvinylpyrrolidone-sodium hyaluronate gels |
| | including Gelclair ™ |

Treatments for dysgeusia and dry mouth

| | |
|---|---|
| 1) | Zinc |
| 2) | Alpha lipoic acid (ALA) |
| 3) | Marinol |
| 4) | Megestrol acetate |
| 5) | Synsepalum dulcificum |
| 6) | Topical clonazepam |
| 7) | Dauloxetine |
| 8) | Other antidepressants |
| 9) | Gabapentin |
| 10) | Pregabalin |
| 11) | Other anti-epileptics |
| 12) | Amisulpride |
| 13) | Capsaicin |
| 14) | Local salivary stimulants |
| 15) | Lemon flavored sprays, ointments, rinses, gels and creams |
| 16) | Saliva substitutes containing substances with an aqueous |
| | component supplemented with calcium, phosphate and fluoride |
| | ions. These would include sprays, ointments, rinses, gels |
| | and [symbol] |

[symbol]

indicates data missing or illegible when filed

TABLE2
Natural mucopolysaccharides

| | |
|---|---|
| 1) | Alginate |
| 2) | Hyaluronic acid |
| 3) | Okra polysaccharide |
| 4) | Honey |
| 5) | Bee propolis |
| 6) | Manuka honey |
| 7) | Aloe vera |
| 8) | CBD (cannabidiol) oil |

TABLE 3

| Criteria | Categories | Pharmacologic agents |
|---|---|---|
| Source | Semi- | Agarose, chitosan, gelatin |
| | natural/natural | |
| | | Hyaluronic acid |
| | | Various gums (guar, hakea, xanthan, gellan, carragenan, |
| | | pectin and sodium alginate) |
| | Synthetic | Cellulose derived |
| | | [CMC, thiolated CMC, sodium CMC, HEC, HPC, HPMC, MC, |
| | | methylhydroxyethylcellulose] |
| | | Poly(acrylic acid)-based polymers |
| | | [CP, PC, PAA, polyacrylates, poly(methylvinylether-co- |
| | | methacrylic acid), |
| | | poly(2-hydroxyethyl methacrylate), poly(acrylic acid-co- |
| | | ethylhexylacrylate), |
| | | poly(methacrylate), poly(alkylcyanoacrylate), |
| | | poly(isohexylcyanoacrylate), |
| | | poly(isobutylcyanoacrylate), copolymer of acrylic acid |
| | | and PEG] |
| | | Others |
| | | Poly(N-2-hydroxypropyl methacrylamide) (PHPMAm), |
| | | polyoxyethylene, |
| | | PVA, PVP, thiolated polymers |
| Aqueous solubility | Water-soluble | CP, HEC, HPC (waterb 38 8C), HPMC (cold water), PAA, |
| | | sodium C:MC, |
| | | sodium alginate |
| | Water-insoluble | Chitosan (soluble in dilute aqueous acids), EC, PC |
| Charged agents | Cationic | Aminodextran, chitosan, dimethylaminoethyl |
| | | (DEAE)-dextran, trimethylated |
| | | Chitosan |
| | Anionic | Chitosan-EDTA, CP, CMC, pectin, PAA, PC, sodium |
| | | alginate, sodium CMC, |
| | | xanthan gum |
| | Non-ionic | Hydroxyethyl starch, HPC, polyethylene oxide), PVA, |
| | | PVP, scleroglucan |
| Bioadhesive force | Covalent bonded | Cyanoacrylate |
| [symbol] ype | | |
| | Hydrogen bond | Acrylates [hydroxylated methacrylate, poly(methacrylic |
| | | acid)], CP, PC, PYA |
| | Electrostatic | Chi tosan |
| | interaction | |

[symbol]

indicates data missing or illegible when filed

TABLE 4
Caphosol ™- A mouth rinse designed to moisten, lubricate and clean the oral cavity including the
mucosa of the mouth, tongue and oropharynx. Ingredients include: Disodium phosphate
0.052%, Monosodium phosphate 0.009%, Calcium chloride 0.052%, Sodium chloride 0.569%
and Purified water. -commercial product
MuGard ™ -Oral Mucoadhesive containing purified water, glycerin, benzyl alcohol, sodium
saccharin, Carbomer Homopolymer A, potassium hydroxide, citric acid, polysorbate 60 and
phosphoric acid. -commercial product
Prothelial ™- Polymerized cross-linked sucralfate malate paste-commercial product
Sucralfate slurry-commercial and generic product
Sucralfate-commercial product
Viscous lidocaine-commercial product and generic
Magic mouthwash-contains a variety of ingredients that may include magnesium aluminum
hydroxide, viscous lidocaine, and diphenhydramine-commercial product and compounded
product
PTA (polymyxin, tobramycin, amphotericin B) antimicrobial paste-commercial product
BCoG (bacitracin, clotrimazole, gentamicin) antimicrobial paste
Palifermin-commercial product, not topical
Nepidermin (brand name Easyef), also known as recombinant human epidermal growth factor
(rhEGF)-commercial product
Palifermin- commercial product, not topical
Innovation Pharma's Brilacidin-OM, an oral rinse version of the company's defensin-mimetic
Brilacidin, a novel synthetic, non-peptidic small molecule shown to have a dynamic mechanism
of action that demonstrates antibiotic, anti-inflammatory and immunomodulatory properties.-
commercial product, in development
Validive ® (clonidine Lauriad ®) is a mucoadhesive tablet based on our Lauriad ® technology that
delivers high concentrations of an anti-inflammatory active principle (clonidine) directly in the
oral cavity, the site of irradiation in the treatment of head and neck cancer.- commercial
product, in development
AG013 delivers the therapeutic molecule Trefoil Factor 1, part of a class of peptides that help
protect and repair gastrointestinal tissue, to the mucosal tissues in the oral cavity- commercial
product, in development
Antioxidants including but not limited to Glutamine, Oral Zinc, Vitamin E, N-Acetyl-Cysteine
(NAC) and Superoxide Dismutase Mimetics
Iseganan antimicrobial mouthwash
Misoprostol mouthwash
Smad7 inhibitors
TGFb inhibitors
NkB inhibitors
Other forms of topical clonidine
Dusquetide- an innate immune inhibitor
Anti-chemokine ligand 9 (CXCL9)
Specific caspase-3 inhibitors
Probiotics including Lactobacillus species
Chlorhexidine
Glucagon-Like Peptide-2 (GLP-2) analogs
Topical morphine-compounded product
Topical clonazepam- compounded product
Topical doxepin- compounded product

Yet other anti-mucositis agents are known in the art. In some therapeutic embodiments, the agents include doxepin mouthwash, topical morphine and morphine mouthwash, topical lidocaine and related drugs, polymerized forms of sucralfate, mucoadhesive substances and barrier coatings of the mucosa. Agents that are advantageously used prophylactically include benzamidine-containing mouthwashes and recombinant human keratinocyte growth factor 1. Aside from mouthwashes, other topical treatments for oral mucositis are typically administered in form of gels and sprays. Anti-mucositis agents may be used alone or in combination of two or more such agents. The methods of the present invention may be used in combination with systemic treatment.

With respect to embodiments of the present invention that are directed to treatment of oral mucositis, the anti-mucositis agents are topically administered to mucosal tissue in a therapeutically effective amount. As persons skilled in the art would appreciate, this amount will vary depending upon the agent itself and other factors which may include one or more of the severity factors of the condition and the general health of the patient. The therapeutic amount may be effective to ameliorate one or more symptoms presented by the patient, e.g., pain, mucosal dryness and oral ulcerations, or even cure the condition.

With respect to aspects of the present invention that are directed to prophylactic uses, the anti-mucositis agent is topically applied to mucosal tissue of a patient suitably prior to an event that is known to induce oral mucositis, e.g., chemotherapy and radiation therapy that are typically used for treatment of cancer, in a prophylactically effective amount. This amount may be effective to delay or inhibit the onset of mucositis or reduce the severity or duration of mucositis or any symptom associated therewith, or even preventing the onset of the condition. Here again, in these aspects of the present invention, persons skilled in the art will appreciate that prophylactically effective amounts will vary depending upon the anti-mucositis agent and depend other factors which may include one or more of the severities of the condition and the general health of the patient.

A common treatment for the pain of mucositis includes numbing of the entire mouth, including the tongue, for example, by application of viscous lidocaine. Advantageously, using the device along with the topical application of lidocaine markedly increased the numbing effect on the tongue even though the device does not cover the tongue. The tongue is normally bathed by circulating saliva and medications are, therefore, easily washed off. With use of the device, the openings for the parotid duct and a number of salivary glands are covered, so saliva pools at the lower parts of the mouth by the effect of gravity. This reduces the amount of saliva bathing the tongue and other parts of the mouth, including the cheeks. In turn, this lessens the effect of circulating saliva from diluting topical medications that are present in the mouth and used for treatment of oral mucositis.

Another aspect of the present disclosure provides a kit for administering a therapeutic agent, for example, an anti-mucositis agent, to the mucosal tissue of an individual. The kit may include a device, as described above, a therapeutic amount of the agent and instructions regarding the method of using the device and the agent to treat the affected mucosal tissue area.

The invention will now be described in terms of the following non-limiting examples.

### Example S

### Example 1

A test was performed to determine the effectiveness of the device by measuring the quantity of orally placed substances that remain in the mouth over time. A study was performed comparing the retention of ethanol alcohol (ETOH) within the mouth over time in a normal volunteer. This study was a comparison of the retention of orally placed ETOH alone, ETOH mixed with alginate gel and a combination of ETOH, and alginate followed by placement of an oral mucositis retention device. The comparative data was obtained as a function of ETOH concentration vs time using linear regression analysis.

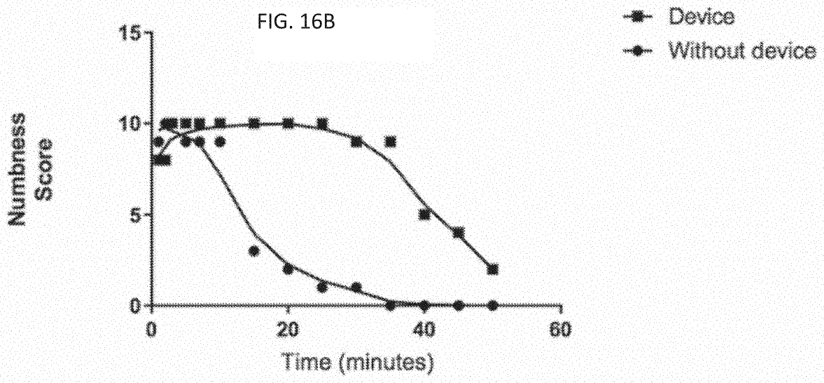

Referring now to FIG. 16A, the data shows that the use of the device resulted in a prolonged retention of the ETOH alginate gel mixture. This was evidenced by a lower elimination rate constant (k) and a prolongation of half-life (T½) of ETOH within the mouth. Without the device, alginate remained in the mouth for only 8 minutes. More specifically, using the device, the ETOH/alginate combination remained in the mouth for at least 37 minutes (when the study was concluded). A marked increase in area under the curve (AUC) was produced by the device. The AUC for ETOH alone was 24.27 ppm*seconds, for ETOH with alginate was 28.35 ppm*seconds and for ETOH plus device was 217 ppm*seconds. Thus, the method of the present invention increased the total amount of alginate that remained on the mucosal surface almost 8 times the amount without the device.

### Example 2

A subject's perception of tongue numbness, per the application of lidocaine with and without the device, was measured. More specifically, a normal subject, i.e., one with normal mouth function, participated in two studies. In the first study, the subject placed 15 mL of 2% viscous lidocaine in the mouth and swished the substance within the mouth for 1 minute. After 1 minute, the viscous lidocaine was expectorated. The subject then determined the degree of numbness of the tongue, at periodic intervals. The degree of numbness was determined by the subject using a Numbness scale from 1 to 10. This scale is a modification of a standard visual analogue pain scale, where a score of 0=no numbness, a score of 5=moderate numbness and a score of 10=extreme numbness. In the second study, the subject placed 15 mL of 2% viscous lidocaine in the mouth and swished the substance within the mouth for 1 minute. After 1 minute, the viscous lidocaine was expectorated, and the device was placed and retained in the mouth.

Referring now to FIG. 16B, curve fitting was applied to generate data from the

Numbness score using the Prism program (GraphPad Software, San Diego, Calif.,). The curve demonstrates that, with and without the device, the peak degree of numbness (Numbness score=lO) occurred rapidly. Without the device, however, peak numbness remained for 9.7 minutes, while peak numbness was present for 34.2 minutes with the device in place. Furthermore, both the area under the curve of numbness score over time and the time for the numbness to reach ½ of its prior level (numbness halftime) were markedly increased when the device was used.

Mouthguard Use for Recurrent Aphthous Stomatitis (also known as recurrent aphthous ulcers or canker sores)

Recurrent aphthous stomatitis (also known as recurrent aphthous ulcers or canker sores) is the most frequently occurring ulcerative disease of the lining of the mouth. The clinical term for canker sores is Recurrent Aphthous Ulcer or RAU. As classically described RAUs are painful Shallow ulcers that are round and have a well-defined erythematous margin with a yellow pseudomembrane in their center. RAUs usually occur in healthy persons but also are seen with immunologic diseases. They are typically located on the cheeks, gums, and tongue. Potential causes of RAUs include vitamin deficiences, oral flora, psychosocial stress and immune dysregulation. Although most RAUs are called minor forms and are less than 1 cm in diameter. Minor RAUs spontaneously within 1 week. Severe forms of RAU are larger than 1 cm in diameter, may take more than 30 days to heal, and may cause scarring of the mouth. Up to 50% of the population is affected by RAUs.

A variety of medications are used to treat RAUs. Topical medications that are applied to the ulcers include emollients such as Zilactin Cold Sore Relief Gel or Orabase Oral paste. Topical antiseptic agents such as topical, intralesional, and systemic corticosteroids, azathioprine, dapsone, colchicine, pentoxifylline, oral tetracycline, thalidomide, and chlorhexidine are also recommended. Stronger oral topical treatments for RAU include corticosteroids and antibiotics. In additon, similar to treatment of oral mucositis in cancer patients, Magic Mouthwash, a compounded medication containing the local anesthetic lidocaine can be used for treatment of RAUs. Since topical oral medications are eliminated from the oral cavity due to mouth movements, salvary dilution and swallowing, their efficacy for treating RAUs may be compromised. Mouth Guard has the potential to improve the effectiveness of these treatments by prolonging their retention and increasing their concentration inside of the mouth. This in turn should amplify the effects of these topical medications. Since Mouth Guard has the effect of increasing the amount and time that oral topical medications stay inside of the mouth, we hereby extend the use of Mouth Guard to include RUA.

Mouth Guard Use for Oral Chronic Graft versus Host Disease

Chronic graft-versus-host disease (cGVHD) is a serious and relatively common complication that occurs in patients undergoing allogeneic hematopoietic stem cell transplantation (HSCT), (also know as bone marrow transplantation (BMT). Multiple body organs can be affected. cGVHD results from an immunologic reaction from the donor cells that causes these to mistake normal recipient organs as antigenic tissue. The oral cavity is commonly affected and oral cGVHD may be seen either alone or in combination cGVHD in other organs such as the skin and liver. Common symptoms of oral cGVHD include pain, mouth discomfort and sensitivity with eating and drinking, xerostomia and decreased range of motion of the mouth, particularly with opening the mouth. Problems with nutrition, tooth decay and overall quality of life are also associated with oral cGVHD. Oral cGVHD is an inflammatory condition of the lining (mucosa) of the mouth causing redness, soreness, and mouth pain. On examination, oral cGVHD may present as lacy white lines (striations) and plaque-like changes are commonly present on the buccal mucosa or the dorsum or sides of the tongue. Oral mucosal erosions/ulcerations consist of raw appearing or erythematous painful and sensitive areas of the mucosa. Ulcerations due to cGVHD are often extremely sensitive and painful. These may affect the ability of patients to maintain adequate nutrition.

Common treatments used for oral cGVHD are shown in Table 5

TABLE 5
Topical Medications Used for the Management of Oral Mucosal cGVHD

| Therapeutic Options | | | Instructions for use |
|---|---|---|---|
| Corticosteroids | Solution | Dexamethasone | Keep solution in mouth for 4- |
| | | 0.1 mg/ml (5 ml) | 6 minutes without |
| | | Budesonide 0.3- | swallowing. |
| | | 0.6 mg/ml (10 ml) | Wait 10-15 minutes before |
| | | Prednisolone | eating or drinking. |
| | | 3 mg/ml (5 ml) | Repeat up to 4-6 times per |
| | | Triamcinolone 1% | day. |
| | | (5 ml) | |
| | Gel, cream, and | Flucinonide | Apply it directly over the |
| | ointment | 0.5% | lesions 2-4 times per day |
| | | Clobetasol | |
| | | 0.05% | |
| | | Triamcinolone | |
| | | 0.1-0.5% | |
| Calcineurin inhibitors | Solution | Tacrolimus | Keep solution in mouth for 4- |
| | | 0.1 mg/ml (5 ml) | 6 minutes without |
| | | Cyclosporine | swallowing. |
| | | | Repeat up to 4-6 times daily |
| | Ointment | Tacrolimus 0.1% | Apply directly over the |
| | | | lesions 2-4 times daily |

| | |
|---|---|
| Oral phototherapy | Methoxypsoralen 3 mg/kg + UVA light |
| | 0.5 J/cm² |
| | Photobiomodulation (formerly |
| | called low level laser therapy) |
| Antimetabolite and | Azathioprine (solution and gel) |
| immunosuppressive | 5 mg/cm³ |
| agents | Thalidomide (solution and |
| | ointment) |

Supportive treatments for oral cGVHD are shown in Table 6.

TABLE 6
Topical Supportive Therapies for Oral cGVHD (1, 17, 40).

| | | |
|---|---|---|
| Pain | Topical | Lidocaine 2% (solution) |
| | anesthetics | Tetracaine, benzocaine |
| | CO₂-laser | 1 W for 2-3 seconds/1 mm² |
| | Photo | |
| | biomodulation | |

| | |
|---|---|
| Xerostomia/hyposalivation | Artificial saliva, Dentifrices, mouth |
| | rinses, gel, gums |
| | Electro-stimulation, Photo |
| | biomodulation |
| Caries and periodontal | Fluorides (dentifrices, varnish, gel) |
| disease | Oral prophylaxis and hygiene encourage |

Oral topical therapies such as mouthwashes, gels and rinses only remain in the oral cavity for a short period of time. They are swished in the mouth for between 30 seconds and six minutes and are then swallowed or spit out. Because the efficacy of these treatments is dependent on the medication that sticks to the oral mucosa after spitting or swallowing the medication, topical therapies have a noticeably short duration of action. The medication that does stay in the mouth after spitting or swallowing is also rapidly removed from the oral cavity by dilution by the saliva. Removal of topical medications from the oral cavity is also facilitated by movements of the mouth and ongoing swallowing.

Oral topical therapies have variable efficacy for cGVHD of the mouth. Because these treatments are often not effective, oral treatment device 200 represents a potential method to improve these treatments. The oral treatment device 200 would allow topical treatments to stay within the oral cavity at a higher concentration for longer periods of time. To test this hypothesis, four patients with cGVHD of the mouth that had recurrent ulcerations and severe mouth pain were given Mouth Guard samples to use together with their oral topical treatments. Three of four of these patients reported that they experienced marked improvement in their mouth pain and other symptoms of oral cGVHD.

Based on oral treatment device's 200 mechanism of action (increasing the amount and duration that oral topical medications stay in the mouth) and our clinical experience, we extend the use of oral treatment device 200 to include cGVHD.

Use of the oral treatment device in accordance with one or more of the above embodiments (such as device 200) can be used to enhance the absorption of sublingually delivered medications. Placement of a drug under the tongue to optimize its absorption into the body is called sublingual drug delivery. When drugs are placed sublingually, they are rapidly absorbed into the circulation. This occurs because the area under the tongue is lined with venous structures that drugs that allow medications to move out of the sublingual space and directly into the bloodstream. By comparison, drugs that are administered in the form of capsules or tablets (known as the oral route of administration) are partially broken down in the intestinal tract and then slowly absorbed into the bloodstream. Thus, sublingually administered medications are more rapidly and efficiently delivered to the organs where effects take place.

There are several instances where the sublingual route of administration of a medication is preferred over the oral route. Some medications, such as nitroglycerin, (used to dilate the coronary arteries during episodes of chest pain), need to have a rapid action for their effect. This is one of the reasons why nitroglycerin is given under the tongue. In addition, when nitroglycerin is taken by mouth, it is mostly broken down in the gastrointestinal tract before it ever enters the bloodstream. Because of this, orally administered nitroglycerin cannot efficiently improve the blood flow to the heart, while sublingually administered nitroglycerin can.

Other medications are administered with the sublingual route when patients are unable to use their gastrointestinal tract. The sublingual route of administration of medications can be effective for patients with nausea, blockages in the intestines or other organs and/or problems with the intestinal absorption of medications. Recently, a sublingual form of ondansetron hydrochloride, a medication that is used for nausea and vomiting has been developed. In this case, the sublingual medication is delivered as a fast absorbing mucosally adherent polymer. Sublingual ondansetron is an attractive alternative to giving ondansetron as a pill or capsule because aa patient with nausea and vomiting would be unable to swallow and keep down a pill or capsule but could hopefully keep the medication under their tongue long enough for absorption into the bloodstream.

Rapid absorption of medications is an also an advantage for some treatments for Parkinson's Disease. Some sublingual forms of anti-Parkinsonian medications such as apomorphine hydrochloride, delivered as a film that is placed under the tongue, are now on the market. Due to their rapid absorption and effects, anti-anxiety medications are also administered in a sublingual form.

Sublingual Dosing of Cannabidiol (CBD)

Use of cannabis for medical purposes has been gaining in momentum and popularity. Medical cannabis has been used for a variety of conditions. Cannabinoids, the active ingredients contained within the cannabis plant, work on specific neuroendocrine receptors within the body called the endocannabinoid system. Although the cannabis plant contains about 113 different cannabinoids, most research has been performed on tetrahydrocannabinol-9 (THC) and cannabidiol (CBD).

THC is the component of cannabis that has psychoactive properties and causes the “high” experienced by marijuana users. It also carries the risk of serious side effects including paranoia, anxiety, and memory loss. THC is used in the medical setting for pain, difficulty sleeping and for nausea and vomiting. THC and medical marijuana are now used as a treatment for patients having nausea and vomiting as side effects of cancer therapy.

CBD does not disturb brain function, produce euphoria, or cause the severe side effects in the brain that occur with the use of THC. A body of scientific evidence now exists on the biochemical properties of CBD that may produce beneficial effects for patients with a variety of diseases. Most commonly, CBD is used in the clinical setting for patients with chronic pain, depression and anxiety. CBD also has anti-inflammatory effects and may represent a promising treatment for chronic autoimmune diseases including Crohn's Disease and rheumatoid arthritis. CBD also has been shown to control some forms of epilepsy. One challenging aspect of using CBD as a medication is that it is poorly absorbed and readily broken down (metabolized) by the body. For example, when CBD is taken orally, only about 5% of the active ingredient is absorbed. This means that is a patient consumes 100 mg of CBD as an edible or capsule, only 5 mg of CBD gets into the body to perform a medical function. To improve this situation, CBD is mixed in oil such as medium chain triglycerides from coconut oil, grape seed or hemp oil is administered with a medicine dropper under the tongue. It has been estimated that between 13 and 19% percent of active CBD is absorbed when CBD oil is administered sublingually (under the tongue). At present, sublingual dosing of CBD oil is the most used form of CBD. Patients using this sublingual method of administration of CBD oil are instructed to try to keep the medication under the tongue for at least 60 seconds. They are also told that they can keep the CBD oil under their tongue longer, better absorption of the CBD oil will take place and the treatment will be more effective. However, keeping CBD oil under the tongue for more than a short time is difficult, and much of the sublingual CBD actually is swallowed.

The oral treatment device 200 can be used to enhance the sublingual absorption of CBD oil. To test this, we have used the Oral Mucosal Absorption Test (OMAT) to measure how rapidly substances that are placed under the tongue leave the sublingual space. The loss of substances from the sublingual space is an indirect measure of how rapidly these substances are absorbed into the bloodstream. Our studies show that oral treatment device 200 increased the speed that sublingually administered alcohol moves out of the sublingual space, suggesting that oral treatment device 200 sped up the process of absorption of these substances. Furthermore, our studies show that when oral treatment device 200 is placed into the mouth after sublingual administration of alcohol, the alcohol was more efficiently absorbed into the bloodstream. In our studies, the mixture of CBD oil and alcohol that was administered sublingually also moved more rapidly and efficiently out of the sublingual space (again presumably into the bloodstream) when the oral treatment device 200 device is placed into the mouth after sublingual administration of the mixture. Oral treatment device 200 enhances the absorption of sublingually administered medications, including CBD oil by several mechanisms. First, oral treatment device 200 covers some of the sublingual glands, reducing the rapid dilution of the sublingually administered medication. Second, just as oral treatment device 200 works for topically applied medications in other parts of the mouth, dilution of the sublingual medication is decreased by blocking the ostia of the parotid glands, slowing down the salivary dilution of the medication. Third, oral treatment device 200 holds the mouth still after placement of medications under the tongue. This allows the medications to stay in place for a longer duration, allowing more time for the medication to be absorbed into the bloodstream. Finally, because of its unique design, when the oral treatment device 200 device is inserted into the mouth after placing a medication under the tongue, a protected sublingual compartment is created. This allows the sublingual medication to remain within a confined, protected space under the tongue. If the medication remains within this space, its absorption into the bloodstream is more efficient. In the case of medications such as CBD that are better absorbed sublingually than in the intestinal tract, the sublingual process of absorption will be further enhanced when oral treatment device 200 is used for this purpose.

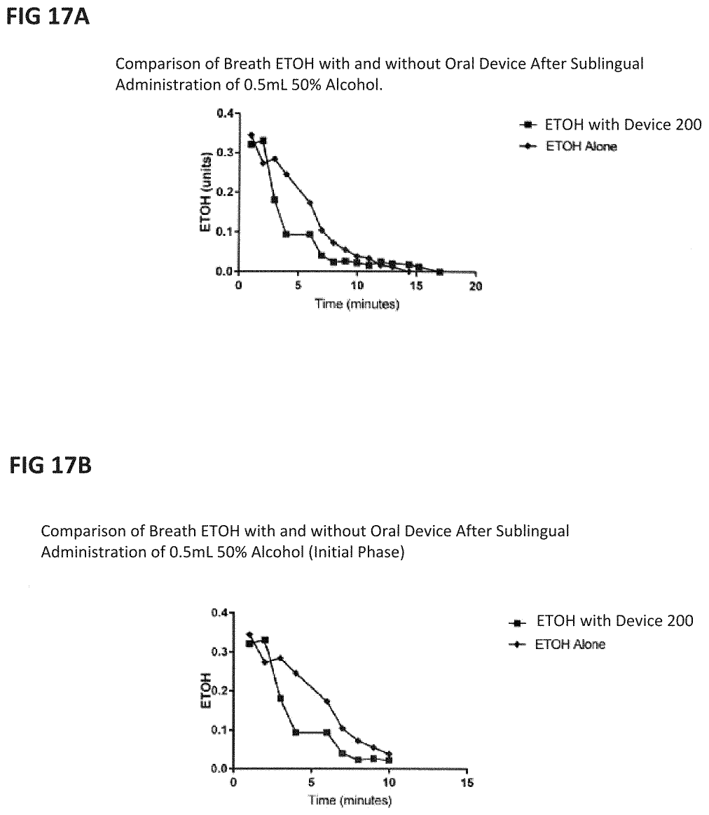

FIGS. 17A and 17B demonstrate the results of experiments showing the effect of the oral treatment device 200 on sublingually administered substances. The study shows the use of the oral treatment device 200 enhances the sublingual absorption of substances placed under the tongue. Thus, oral treatment device 200 represents a new approach to improving the efficacy of sublingual medications. FIGS. 17A and 17B show the results of a study measuring ETOH levels in the breath after it is placed under the tongue using a medicine dropper. For this study, alcohol was placed under the tongue with and without the insertion of the oral treatment device 200 device after the sublingual administration. In FIG. 17A, the study demonstrates how sublingual alcohol exits the sublingual space much more rapidly and completely when the Oral Device 200 is used. This implies that the use of Device 200 with sublingually administered substances results in more rapid and efficient absorption. In FIG. 17B, the graph shows that Device 200 accelerates the elimination of ETOH from the sublingual space. This suggests that the Device 200 speeds up and enhances the absorption of sublingual medications. Measurement of the areas under the two curves show that the Device 200 increased the sublingual absorption by 35%. Sublingual medications depend on rapid, early absorption to be effective. Thus, the oral treatment device 200 is shown to accelerate the loss of alcohol from the sublingual space. The study suggests that oral treatment device 200 enhances the sublingual absorption of alcohol.

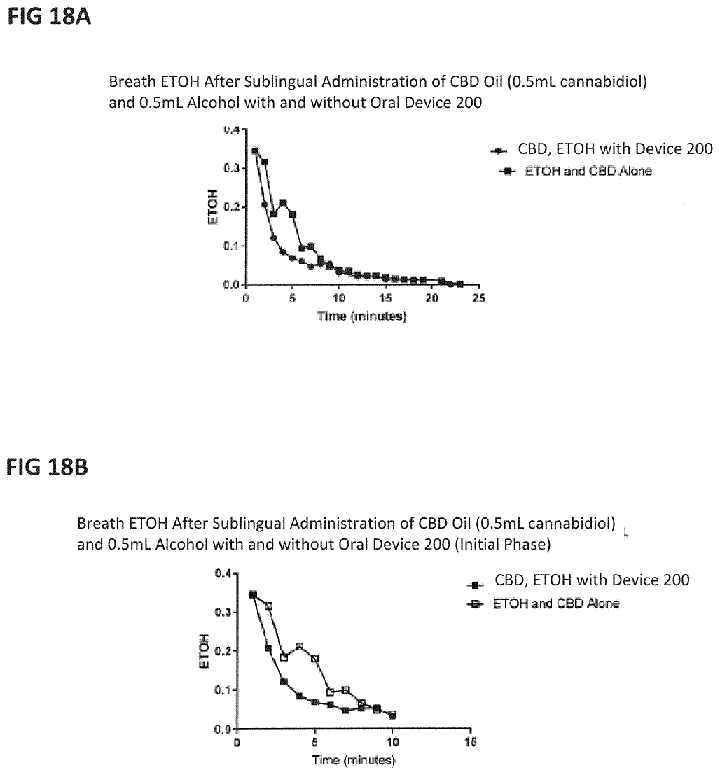

FIGS. 18A and 18B show the results of a study measuring ETOH levels in the breath after a mixture of CBD oil and alcohol is placed under the tongue using a medicine dropper. For this study, alcohol was placed under the tongue with and without the insertion of the oral treatment device 200 after the sublingual administration of the CBD oil and alcohol mixture.

In FIG. 18A, the insertion of the Device 200 in the mouth after sublingual administration of CBD oil with an alcohol marker shows that with the Device 200, a smooth and rapid decline in measured ETOH levels occurs. By comparison, without the Device 200, more ETOH remains in the sublingual space and ETOH levels decline more slowly and erratically. This suggests that the Device 200 enhances the absorption of the sublingual medications, including CBD oil. In FIG. 18B, when the Device 200 is used, the levels of ETOH mixed with CBD oil and placed into the sublingual space declines rapidly and smoothly. By comparison, without the Device 200, ETOH mixed with CBD oil and placed into the sublingual space declines more slowly and erratically. This suggests that the Device enhances the absorption of the sublingual medications, including CBD oil. Thus, the results of the study suggest that oral treatment device 200 enhances the absorption of sublingual CBD oil.

Use of oral treatment device 200 for Patients with Xerostomia, Also Known as Dry Mouth. Xerostomia is a chronic condition that is characterized by the feeling of dryness of the inside of the mouth. There are many potential causes of xerostomia. Some of these conditions occur because of a decrease in saliva production. This most commonly occurs as a part of the normal aging process. In addition, a variety of medications can cause xerostomia. These include anticoagulants, and treatments for depression, high blood pressure, HIV infection and diabetes. Other medications that can cause xerostomia include multivitamins, nutritional supplements, non-steroidal anti-inflammatory agents, and inhalers. Common treatments for xerostomia are aimed at increasing the production and flow of saliva or lubricating the inside of the mouth. Both types of medications aim to reduce the sensation of dryness of the mouth. The most common treatments for xerostomia include mouthwashes, rinses and gels that are swished inside of the mouth (oral topical therapies). These medications include mucosal lubricants, saliva substitutes, and saliva stimulants. Saliva stimulants and substitutes (gels, mouthwashes, and toothpastes) are available over the counter. Oral sprays that mimic natural saliva that contain mucin, electrolytes, oils, betaine, xylitol, methylcellulose, xanthan gum, buffered Profylin gel64, maltose and anticholinesterase physostigmine have also been studied for xerostomia. As with other conditions, oral treatment device 200 can be inserted into the mouth after topical treatments for xerostomia. The efficacy of these treatments should be enhanced by increasing the contact time of these treatments in patients with xerostomia, allowing for increased efficacy of the treatments.

Use of oral treatment device 200 for Patients with Excessive Saliva Production, Also Known as Hypersalivation. Excessive saliva production and inappropriate handling of saliva that is produced in the mouth is called hypersalivation. The scientific terms for hypersalivation are sialorrhea or ptyalism. Patients with hypersalivation suffer from uncontrollable loss of saliva from the mouth, commonly known as drooling. Normal people in good health person produce between 0.75 and 1.5 liters of saliva per day. However, most people can swallow the amount of saliva that they produce. For patients with hypersalivation, the amount of saliva produced, overwhelms their ability to swallow their saliva. Patients with hypersalivation may develop lip and gum problems, including infections. They may also have socialization problems and embarrassment because of their condition. Patients with this problem may be producing too much saliva. They may also have swallowing problems form neurologic diseases including brain injury and Parkinson's disease. Enlargement of the tongue, difficulty with head control and a variety of other conditions may cause hypersalivation and drooling.

Treatments for hypersalivation include systemic medications that dry the mouth. These have significant side effects. Speech therapists can work with patients to improve this condition. Oral treatment device 200 has promise for patients with hypersalivation and drooling. Oral treatment device 200 can improve these conditions by several mechanisms. First oral treatment device 200 has been engineered to reduce salivary secretions and accumulation. This effect is achieved by inserting oral treatment device 200 inside of the mouth. Portions of the oral treatment device 200 cover some of the salivary glands, reducing the amount of saliva that accumulates inside of the mouth. oral treatment device 200 achieves this effect by an upper portion of the device that partially blocks the ostia (openings) of the parotid glands. In addition, a lower portion of the device partially blocks the some of the sublingual salivary glands. The overall effect of wearing the oral treatment device 200 is to reduce the pooling of saliva within the mouth. Second, wearing oral treatment device 200 stabilizes the mouth and jaw, allowing better control of the mouth muscles and muscles involved with swallowing. This allows for improved handing of salivary secretions.

Use of oral treatment device 200 for Patients with Excessive Mouth Movements, Also Known as Oral Dystonia or Oromandibular Dystonia. Oromandibular dystonia, is a serious condition. Patients with oromandibular dystonia have movements of their face, tongue, and jaw that they cannot control. The condition occurs because muscle contractions in the head and neck regions are occur. These cause the involuntary movements seen in oromandibular dystonia. Patients with oromandibular dystonia suffer from social embarrassment, as well as problems speaking and swallowing. Oromandibular dystonia is seen in a variety of neurologic disease, including Parkinson's disease. It may occur as an irreversible side effect of medications used to treat psychiatric disorders. This condition is called tardive dyskinesia. Treatments for oromandibular dyskinesia include muscle relaxing medications. As with other forms of dystonia, Botulinum toxin A injections into involved muscles may be highly effective.

Insertion of the oral treatment device 200 into the mouth may be beneficial to patients with oromandibular dystonia because oral treatment device 200 stabilizes the mouth cavity. This in turn can reduce involuntary mouth movements. oral treatment device 200 can be used in conjunction with relaxation techniques in these patients, giving them additional support within the oral cavity while they are performing the relaxation exercises.

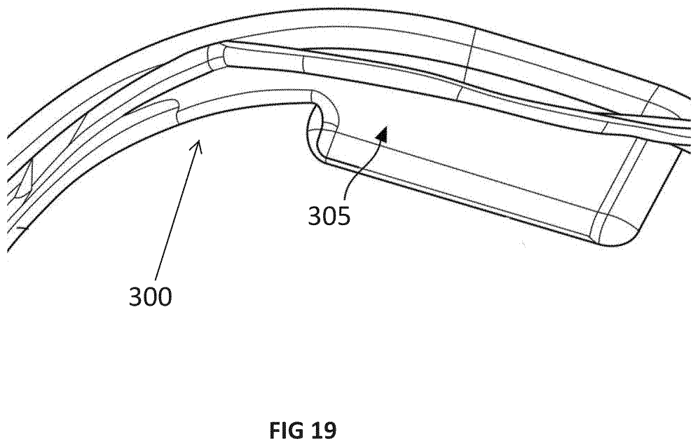

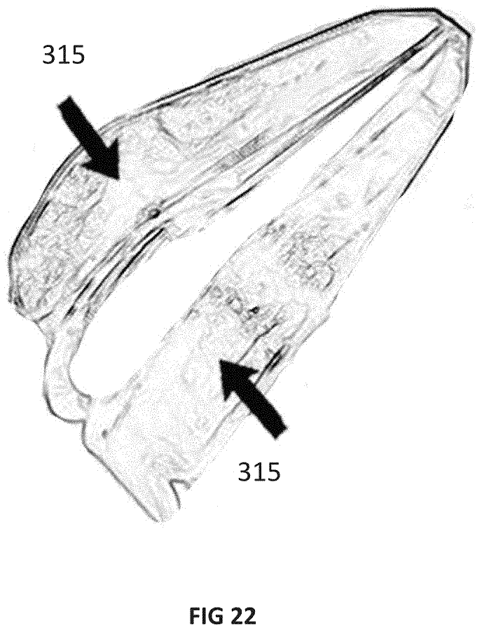

Referring now also to FIGS. 19 through 24, there are shown various oral treatment devices. A method of enhancing absorption of a therapeutic agent sublingually in a person, the method comprising: administering a therapeutically effective amount of the therapeutic agent sublingually in a person and inserting, in a mouth of the person, a device comprising an oral retention portion that is suitably shaped to be retained in an oral cavity for a predetermined treatment period to enhance the absorption of the therapeutic agent sublingually.

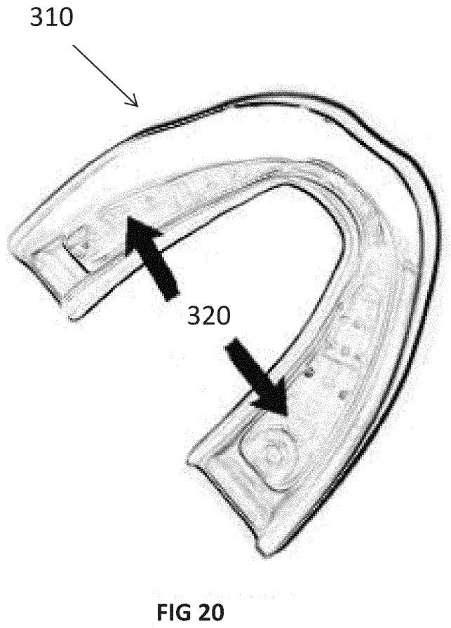

The inserted device has a portion to bite down on between the upper and lower teeth to stabilize the mouth after application of the sublingual medication. This biting portion of the device can take the form of a phalanx 305 that is held between only a small portion of the teeth on the lateral or medial side of the mouth (FIG. 19, 300) or an arcuate shape (FIG. 20, 310) corresponding to a dental arch of the person that will held by the upper and/or lower teeth (see 320).

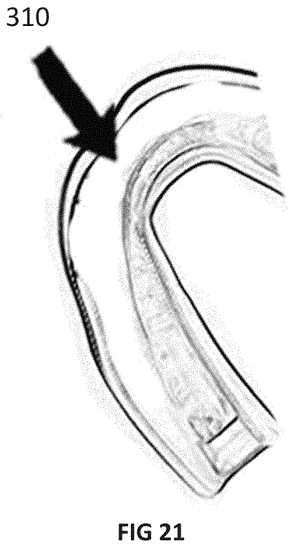

The inserted device may also have an outing covering portion (wing) that fits between the outside of the teeth and gums and the inside of the cheeks that extends upward from the device (FIG. 21, 310) and/or downwardly from the exterior surface of the fitting portion or extending both upwardly and downwardly from the fitting portion of the device (FIG. 21, 320).

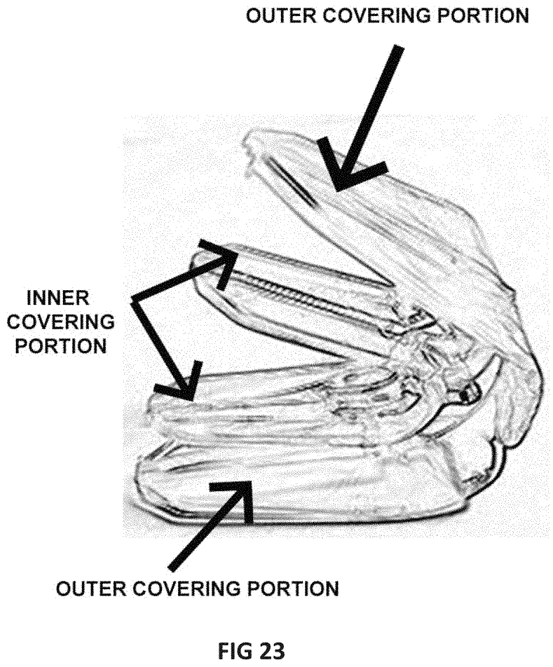

The inserted device may also have an inner covering portion (wings) that fits between the inside of the teeth and roof and floor of the mouth (FIG. 23).

The inserted device stabilizes and isolates the sublingual compartment of the mouth to allow for improved absorption of sublingually. This accomplished by the parts of the device forming a mechanical barrier to contain substances within the sublingual space. The components of the containment of substances within the sublingual space include side portions of the device that create a wall-like effect to reenforce the barrier of the sublingual space formed by the teeth. The combination of side portions of the device, together with the lower teeth, prevent the leakage of the sublingually administered medications out of the sublingual space. Additionally, the components of the device help to keep the mouth closed by providing a biting-down surface for holding the upper and lower teeth together in a closed position. When the mouth is closed, the lower portion of the tongue provides a roof over the sublingual space. This helps to contain the sublingually applied medication within the sublingual space. The device may also have a portion that covers the area beneath the tongue and floor of the mouth inside of the teeth. This is a winglike portion of the device with similar thin structure and fits under the tongue (as shown in FIGS. 11 and 12). As shown in the figures, the tongue can be slipped into combined portions 850 and 880, thus providing coverage of the lateral tongue, the ventral tongue, and the mucosa of the floor of the mouth inside of the teeth.

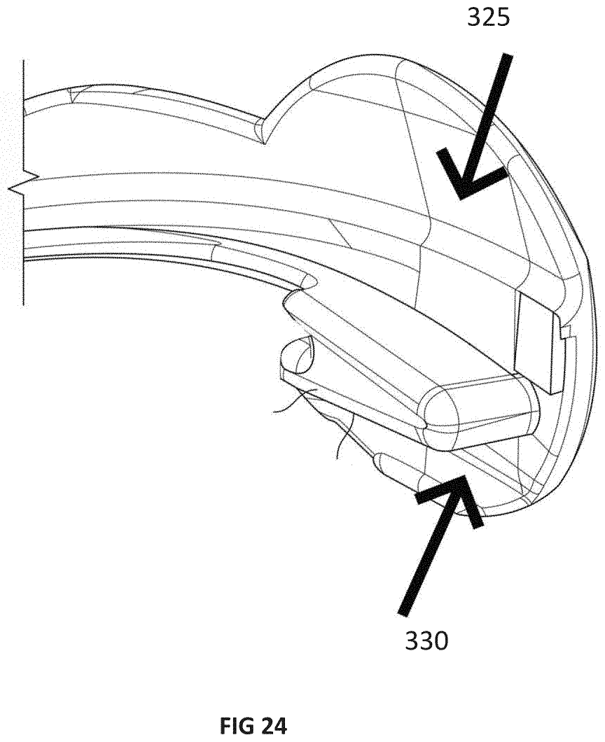

The inserted device may have covering portions (wings) that cover the ostia of the parotid glands (FIG. 24, 325) and/or portions of the sublingual glands to reduce the salivary dilution of the sublingually administered medication (FIG. 24, 330).

In other aspects the device is further defined to have a fitting portion having an arcuate shape corresponding to a dental arch of the person, the fitting portion has an interior surface facing the person's teeth and has an exterior surface facing the person's check and gums, and the fitting portion further configured to have a front middle section with ends on either side of the front middle section such that the ends of the fitting portion are positioned towards the person's back teeth, an upper bite flange and a lower bite flange extending from the interior surface of the fitting portion forming a bite wedge along each end of the fitting portion, a covering portion extending upwardly and downwardly from the exterior surface of the fitting portion, the covering portion further extending from the middle section toward each end of the fitting portion, such that the covering portion defines an upper wing portion positioned at each end of the fitting portion, and a lower wing portion positioned at each end of the fitting portion, and wherein each lower wing portion further includes a V-shaped notch being positioned adjacent each bite wedge towards the middle section such that wherein the lower surface edge along the middle section of the fitting portion extends arcuately towards each lower wing portion the V-shaped notch defines a gap between the lower surface edge and a lower tab extending from the lower bite flange.

In other aspects, the device further is defined to have each upper wing portion having an upper profile surface configured to include a first upper arcuate surface edge tapering upwardly to an uppermost wing edge that extends to a position higher than the upper surface edge along the middle section of the fitting portion and the upper profile surface further configured to include a second upper arcuate surface edge tapering downwardly from the uppermost wing edge to the end of the fitting portion, and each lower wing portion having a lower profile surface configured to include a first lower arcuate surface edge tapering downwardly to a lowermost wing edge that extends to a position lower than the lower surface edge along the middle section of the fitting portion, and the lower profile surface further configured to include a second lower arcuate surface edge tapering upwardly from the lowermost wing edge to the end of the fitting portion, and wherein the upper wing portion and the lower wing portions having a defined outer surface shape to cover the mucosal tissue of the person's mouth, and wherein the at least one covering section is more flexible than the fitting portion.

The invention is directed in various embodiments, wherein administering the therapeutically effective amount of the agent comprises administering a therapeutically effective amount of an cannabidiol (CBD) oil, cannabinol such as tetrahydrocannabinol (THC), anti-emetic medication such as ondansetron hydrochloride, anti-anxiety medication, a CBD spray, anti-epileptic medication, a pain-relieving medication such as an opioid medication, methadone, or other sublingual substances that are used for medicinal purposes.

Although aspects of the present invention herein have been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

I claim:

1. A method of treating chronic graft-versus-host disease (cGVHD) in a person, the method comprising:
administering a therapeutically effective amount of a therapeutic agent configured to treat oral mucosal cGVHD sublingually in a person;
inserting, in a mouth of the person, a device comprising an oral retention portion that is suitably shaped to be retained in an oral cavity for a predetermined treatment period to enhance the absorption of the therapeutic agent sublingually; and
wherein the device is further defined to have a fitting portion having an arcuate shape corresponding to a dental arche of the person, the fitting portion has an interior surface facing the person's teeth and has an exterior surface facing the person's check and gums, and the fitting portion further configured to have a front middle section with ends on either side of the front middle section such that the ends of the fitting portion are positioned towards the person's back teeth, an upper bite flange and a lower bite flange extending from the interior surface of the fitting portion forming a bite wedge along each end of the fitting portion, a covering portion extending upwardly and downwardly from the exterior surface of the fitting portion, the covering portion further extending from the middle section toward each end of the fitting portion, such that the covering portion defines an upper wing portion positioned at each end of the fitting portion, and defines a lower wing portion positioned at each end of the fitting portion, and each lower wing portion further includes a v-shaped notch being positioned adjacent each bite wedge towards the middle section such that wherein the lower surface edge along the middle section of the fitting portion extends arcuately towards each lower wing portion the v-shaped notch defines a gap between the lower surface edge and a lower tab extending from the lower bite flange.

2. The method of claim 1, wherein administering the therapeutically effective amount of a therapeutic agent configured to treat oral mucosal cGVHD includes administering a therapeutically effective amount of a corticosteroid in a solution, gel, cream or ointment.

3. The method of claim 2, wherein the therapeutically effective amount of the corticosteroid includes a solution of one or more of the following: dexamethasone, budesonide, prednisolone, and triamcinolone.

4. The method of claim 1, wherein administering the therapeutically effective amount of a therapeutic agent configured to treat oral mucosal cGVHD includes administering a therapeutically effective amount of a calcineurin inhibitor.

5. The method of claim 4, wherein the therapeutically effective amount of the calcineurin inhibitor includes a solution or ointment containing one or more of the following: tacrolimus and cyclosporine.

6. The method of claim 1, wherein administering the therapeutically effective amount of a therapeutic agent configured to treat oral mucosal cGVHD includes administering a therapeutically effective amount of a topical anesthetic.

7. The method of claim 6, wherein the therapeutically effective amount of the topical anesthetic includes a solution of one or more of the following: lidocaine, tetracaine, and benzocaine.

8. The method of claim 1, wherein administering the therapeutically effective amount of a therapeutic agent configured to treat oral mucosal cGVHD includes administering a therapeutically effective amount of one or more of the following: fluorides, oral prophylaxis, artificial saliva, and dentifrice.

9. A method of enhancing absorption of a therapeutic agent sublingually in a person, the method comprising:
administering a therapeutically effective amount of a therapeutic agent configured to treat oral mucosal cGVHD sublingually in a person;
inserting for a predetermined treatment period a device in an oral cavity of the person, wherein the device comprising an oral retention portion that is configured to be retained in the oral cavity from the predetermined treatment period and further configured to enhance absorption of the therapeutic agent sublingually.

10. The method of claim 9, wherein the device is configured to have a fitting portion having an arcuate shape corresponding to a dental arche of the person, the fitting portion has an interior surface facing the person's teeth and has an exterior surface facing the person's check and gums, and the fitting portion further configured to have a front middle section with ends positioned towards the person's back teeth, the fitting portion further configured to have an upper bite flange and a lower bite flange extending from the interior surface of the fitting portion forming a bite wedge along each end of the fitting portion, a covering portion extending upwardly and downwardly from the exterior surface of the fitting portion, the covering portion further extending from the middle section toward each end of the fitting portion, such that the covering portion defines a rounded upper wing portion positioned at each end of the fitting portion, and defines a rounded lower wing portion positioned at each end of the fitting portion, and each rounded lower wing portion further includes a v-shaped notch being positioned adjacent each bite wedge towards the middle section such that wherein the lower surface edge along the middle section of the fitting portion extends arcuately towards each lower wing portion the v-shaped notch defines a gap between the lower surface edge and a lower tab extending from the lower bite flange.

11. The method of claim 10, wherein administering the therapeutically effective amount of a therapeutic agent configured to treat oral mucosal cGVHD includes administering a therapeutically effective amount of a corticosteroid in a solution, gel, cream or ointment.

12. The method of claim 11, wherein the therapeutically effective amount of the corticosteroid includes a solution of one or more of the following: dexamethasone, budesonide, prednisolone, and triamcinolone.

12. The method of claim 10, wherein administering the therapeutically effective amount of a therapeutic agent configured to treat oral mucosal cGVHD includes administering a therapeutically effective amount of a calcineurin inhibitor.

13. The method of claim 12, wherein the therapeutically effective amount of the calcineurin inhibitor includes a solution or ointment containing one or more of the following: tacrolimus and cyclosporine.

14. The method of claim 10, wherein administering the therapeutically effective amount of a therapeutic agent configured to treat oral mucosal cGVHD includes administering a therapeutically effective amount of a topical anesthetic.

15. The method of claim 14, wherein the therapeutically effective amount of the topical anesthetic includes a solution of one or more of the following: lidocaine, tetracaine, and benzocaine.

16. The method of claim 10, wherein administering the therapeutically effective amount of a therapeutic agent configured to treat oral mucosal cGVHD includes administering a therapeutically effective amount of one or more of the following: fluorides, oral prophylaxis, artificial saliva, and dentifrice.

17. A method of treating aphthous stomatitis in a person, the method comprising:
administering a therapeutically effective amount of a therapeutic agent configured to treat aphthous stomatitis sublingually in a person;
inserting, in a mouth of the person, a device comprising an oral retention portion that is suitably shaped to be retained in an oral cavity for a predetermined treatment period to enhance the absorption of the therapeutic agent sublingually; and
wherein the device is further defined to have a fitting portion having an arcuate shape corresponding to a dental arche of the person, the fitting portion has an interior surface facing the person's teeth and has an exterior surface facing the person's check and gums, and the fitting portion further configured to have a front middle section with ends on either side of the front middle section such that the ends of the fitting portion are positioned towards the person's back teeth, an upper bite flange and a lower bite flange extending from the interior surface of the fitting portion forming a bite wedge along each end of the fitting portion, a covering portion extending upwardly and downwardly from the exterior surface of the fitting portion, the covering portion further extending from the middle section toward each end of the fitting portion, such that the covering portion defines an upper wing portion positioned at each end of the fitting portion, and defines a lower wing portion positioned at each end of the fitting portion, and each lower wing portion further includes a v-shaped notch being positioned adjacent each bite wedge towards the middle section such that wherein the lower surface edge along the middle section of the fitting portion extends arcuately towards each lower wing portion the v-shaped notch defines a gap between the lower surface edge and a lower tab extending from the lower bite flange.

18. The method of claim 1, wherein administering the therapeutically effective amount of a therapeutic agent configured to treat aphthous stomatitis includes administering a therapeutically effective amount of a one or more of the following: corticosteroids, azathioprine, dapsone, colchicine, pentoxifylline, oral tetracycline, thalidomide, chlorhexidine, and lidocaine.
